(19) United States
(12) Patent Application Publication
(10) Pub. No.: US 2015/0320889 A1
(43) Pub. Date: Nov. 12, 2015

(54) Vectorised Magnetic Emulsion

(71) Applicant: GUERBET, Villepinte (FR)

(72) Inventors: Caroline Robic, Nogent Sur Marne (FR); Marc Port, Deuil La Barre (FR)

(73) Assignee: GUERBET, VILLEPINTE (FR)

(21) Appl. No.: 14/762,064

(22) PCT Filed: Jan. 23, 2014
(86) PCT No.: PCT/EP2014/051348

§ 371 Date: Jul. 20, 2015

(30) Foreign Application Priority Data
Jan. 23, 2013 (FR) 13 50584

(51) Int. Cl. A61K 49/18

(52) U.S. Cl. CPC A61K 49/1806; A61K 49/1839

(57) ABSTRACT

An oil-in-water nanoemulsion composition for MRI, comprising an aqueous phase, a lipid phase as nanodroplets comprising an oil and magnetic particles based on an iron compound and covered with one or several C8-C22 fatty acids, and a mixture of surfactants at the interface between the aqueous and lipid phases, the mixture of surfactants comprising at least one amphiphilic lipid and at least one amphiphilic targeting ligand.

The invention relates to novel optimized systems of the nanoemulsion type, sometimes called magnetic emulsions and to their use as contrast agents, notably in MRI.

Administration of contrast products contributes to improving the resolution of MRI images obtained by this technique and the accuracy of the diagnostic. Thus, the contrast effect may be enhanced by the presence, in the environment of the organs subject to examination, of various magnetic species, for example paramagnetic, ferromagnetic or super-paramagnetic species.

In the field of diagnostic imaging, a large number of investigations were related to the elaboration of novel contrast products targeting a region of interest in order to provide assistance with the diagnostic of various pathologies, notably of cancer and for thus allowing tracking of the effectiveness of the treatment of these pathologies. For this, the goal of the various research teams, who have worked on these problems, was to obtain images with great quality. This is only achievable by the use of a vectorised contrast agent with high sensitivity.

To this day, these are especially contrast products in the field of nuclear medicine (for example, PET (Positron Emission Tomography)) tracers which have been or which are studied for these problems for tracking treatment, because of the excellent sensitivity of these tracers. These imaging methods have drawbacks, in that the patient is thus exposed to radiations and they require constraining logistics for protecting the patient and for making the radio-tracer (for example via a cyclotron).

Paramagnetic substances comprise certain metals like iron, manganese, gadolinium in an ionic or organometallic state. Ferromagnetic contrast substances generally comprise magnetic aggregate particles of a micrometric or sub-micron size, i.e. not less than 100-200 nm, for example ferrite particles, notably including magnetite (Fe₃O₄), maghemite (γ-Fe₂O₃) and other magnetic mineral compounds of transition elements which behave like permanent magnets. Super-paramagnetic particles are usually very small ferrite particles, notably including magnetite (Fe₃O₄), maghemite (γ-Fe₂O₃) and other magnetic mineral compounds of transition elements, with a size of less than about 100-150 nm.

Unlike ferromagnetic particles, superparamagnetic particles no longer behave like small autonomous magnets because their size is less than a critical value, i.e. they exclusively align with each other in a preferential direction when they are subject to an external magnetic field. Superparamagnetic particles advantageously have an effectiveness density which is higher as compared with ferromagnetic particles. The colloidal solutions of ferromagnetic nanoparticles or ferromagnetic substances in a solvent or water are called “ferrofluids”.

The lack of sensitivity of the MRI imaging technique, however, always poses problems. The images obtained with certain contrast products, such as these superparamagnetic particles, may thus not be sufficient for allowing a rapid and exact diagnostic or for estimating the efficiency of a treatment of a pathology. In order to improve this sensitivity, more concentrated contrast products must theoretically be provided but this may make these products less tolerable for the patient.

The nanosystems to which belong nanoemulsions give the possibility of accumulating contrast agents in this type of structure. Depending on the type of nanosystem used, this accumulation may be more or less significant which has a direct influence on the quality of the obtained images.

If these contrast products are products targeting particular receptors, additional affinity problems of these contrast products towards these receptors and/or of accessibility to these receptors and/or of the density of these receptors may also be posed.

Emulsions of nanoparticles based on iron, of the USPIO (Ultrasmall Super Paramagnetic Iron Oxide) type, have been proposed since a few years, nanoemulsions also sometimes called “magnetic emulsions”, including in certain cases, biovectors allowing them to target particular receptors. These nanoemulsions do not always give the possibility of solving in a satisfactory way the problem of the lack of sensitivity of the MRI technique because the iron loading capacity is limited and their affinity towards targeted receptors is low.

WO 2005/014051 describes emulsions comprising nanodroplets, which may be used as a contrast agent or for releasing a therapeutic agent. These nanodroplets, formed with oil coupled with atoms having a significant number Z and in particular greater than 36 (like yttrium, zirconium, silver or gold) are covered with a lipid layer and are coupled with a biovector. U.S. 2010/0297019 describes droplets comprising a metal core in an oily substance and an outer layer made of an amphiphilic compound. The sensitivity of this contrast product is not sufficient in the case when the targeted pathology gives rise to low expression of receptors.

In Mandal et al (Langmuir, Vol. 21, No. 9, 2005) iron nanoparticle emulsions are described, inter alia consisting of maghemite (γ-Fe₂O₃) and of oleic acid. There also, the described oily phases (in this case octane) in this document do not give the possibility of obtaining a satisfactory nanoemulsion.

In Jarzyna et al (Biomaterials, 30, 6947-6954, 2009) are described iron nanoparticle emulsions notably consisting of magnetite (Fe₃O₄) covered with oleic acid, the oily phase of these emulsions being mainly made from soya bean oil (in this case a polyunsaturated oil. These nanoemulsions are coupled with a fluorophore Cy5.5, a fluorescent agent belonging to cyanines. This type of compound does not give the possibility of vectorising a nanosystem. This just gives the possibility of being able to ascertain that what is seen in imaging is visible in fluorescence. The described nanoemulsions have a reduced iron loading capacity (not more than 15 mmol of iron per liter of emulsion) and therefore do not give the possibility of obtaining MRI images of sufficient quality.

In Senpan et al (JACS, Vol. 3, No. 12, 3917-3926, 2009) iron nanoparticle emulsions are described, notably consisting of magnetite (Fe₃O₄) covered with oleic acid, the oily phase of these emulsions being mainly made from almond oil (also a polyunsaturated oil). Almond oil is further not adapted to a use in intravenous injection and a priori cannot be used in a topical or parenteral administration. These nanoemulsions are vectorised by means of derivatives of biotin. In addition to the fact that the link of this type of compound is complicated to make from an industrial point of view, the nanoemulsions described in this document also have a reduced iron loading capacity (not more than 80 mmol of iron per liter of emulsion) and therefore do not give the possibility of obtaining an optimum sensitivity in MRI.

A larger dose of these emulsions theoretically would give the possibility of obtaining an image of optimum quality, but without any doubt this would result in a saturation of the targeted receptor if this type of emulsion was vectorised. There also, the sensitivity of these contrast products is therefore insufficient and notably in the case when the targeted pathology gives rise to low expression of receptors.

With the intention of developing vectorised nanoemulsions giving the possibility of obtaining MRI images of optimum quality, several problems have been identified:
In order to obtain images of very great quality, emulsions must have the possibility of accumulating in them more than 100, or even more than 120 or more than 140 mmol of iron per liter of emulsion and these emulsions comprising nanoparticles based on iron must have good affinity and good selectivity towards the receptor which they target,
The iron nanoparticles must have optimum solubility in the oily phase making up the emulsion,
The oils used for making these emulsions must be in the pharmacopeias, i.e. their uses are authorized by regulations for use in patients,
These emulsions must be stable over time, i.e. they do not aggregate irreversibly or there is no destruction of the emulsion, for example by coalescence or irreversible aggregation of the nanodroplets making it up.

Further, a surfactant amount of at least about 5% of the composition by weight, is expressed by:
the formation in the composition, in addition to the nanodroplets, of micelles (without any oily core), the withdrawal of which would require for production at an industrial scale hundreds of tons of contrast product, complex and expensive separation and purification steps and therefore a drop in the industrial yield,
the difficulty or even the impossibility of incorporating to the nanodroplets a suitable amount of biological targeting ligands, the cost of which is very high: amphiphilic lipid surfactants have a stronger surfactant power than amphiphilic targeting ligands and will preferentially form the layer around the oil (and/or the layer of amphiphilic lipids is formed from these lipids before the targeting ligands have the time of being integrated within this layer).

Still more specifically:
when the maximum amount of surfactants (a surfactant of the Lipoid® type or the like, an amphiphilic targeting ligand, a pegylated lipid if required) around nanodroplets is attained, the amphiphilic compounds of the solution will rapidly form micelles, and the solution then contains much more micelles than nanodroplets,
the industrial price cost of nanoemulsions is for about 80 to 90% represented by the targeting ligand which is made amphiphilic, it is therefore understood that a loss of targeting ligands generates much too significant industrial over cost,
if the amount of surfactant amphiphilic lipids (non-vectorised compounds) is too large, the amphiphilic targeting ligands cannot be satisfactorily integrated into the amphiphilic layer around the oil, which generates a highly significant loss of affinity and makes the product unsuitable for specific targeting of the pathological territory.

The nanoemulsions according to the invention are biovectorised (by the presence of the targeting ligand) since they are intended for diagnostic molecular imaging. In the prior art, several ways for vectorising emulsions are described. The two main vectorisation methods are the grafting of targeting ligands on one or several compounds of the emulsion after synthesis of the emulsion or grafting of targeting ligands on one or more compounds of the emulsion followed by synthesis of the emulsion comprising this compound and the other constituents (this will then be referred as incorporation of the targeting ligand).

In the case when the choice is made for grafting targeting ligands on compounds of the emulsion after synthesis of the emulsion, impurities are formed during the grafting. This poses a significant problem since these impurities are on the emulsion, it is difficult or even impossible to remove them.

In the case when the choice of incorporating targeting ligands is made, the nanodroplets of the nanoemulsion have, incorporated at the layer formed by the surfactants, one or several specific targeting ligands which will specifically recognize by molecular interaction (target/ligand affinity) the biological target (receptor, enzyme . . . ), the expression of which is modified in the pathological area. These targeting ligands are also designated as “biovectors” or “recognition ligands” by one skilled in the art.

Now, a technical problem which is very difficult to solve is suitably incorporating and with stability over time one or several targeting ligands for molecular imaging, in a sufficient amount in order to obtain specificity of the marking, but not too high in order to avoid too high industrial price cost.

Considering this complex prior art, the difficulty in obtaining vectorised nanoemulsions for MRI both chemically industrialisable and stable, biologically performing is seen and allowing MRI images to be obtained in great quality.

Nanoemulsions comprising superparamagnetic particles, encapsulated in vectorised nanodroplets and solving all the technical problems of the prior art were obtained.

In the following of the text, the terms of “nanodroplet”, “nanoemulsion droplet” or “nano-object” will be equivalent.

In particular, optimized compositions were selected with improved iron loading capacities and comprising sufficient surfactant in order to stabilize the size of the nanodroplets, but not too much in order to avoid insufficient incorporation of the targeting ligands.

In the nanoemulsions according to the invention, the targeting ligand should be able to be accommodated within the oil/water interface, by being anchored in the amphiphilic film/membrane of the surfactants. It is not at all obvious for one skilled in the art to find the right compounds and the correct ratios of amounts between the surfactants, the oil and the targeting ligands, which give the possibility of obtaining nanoemulsions with improved effectiveness in molecular imaging and without a loss of very expensive targeting ligands.

For this purpose, the invention according to a first aspect relates to a composition of an oil-in-water nanoemulsion, notably for MRI, comprising:
from 50 to 90% by weight, advantageously from 60 to 85% by weight, more advantageously from 75 to 85% by weight, even more advantageously from 78 to 82% by weight of aqueous phase,
from 9.5 to 49.5% by weight, advantageously from 9.5 to 39.5% by weight, more advantageously from 14.5 to 24.5% by weight, even more advantageously from 17.5 to 21.5% by weight of lipid phase as nanodroplets comprising:
   an oil, and
   magnetic particles (p) based on an iron compound, said magnetic particles (p) being covered with one or several C8-C22 fatty acids, advantageously C14-C18, more advantageously C16-C18, even more advantageously C18 fatty acids,
from 0.38 to 4.95% by weight, advantageously from 0.5 to 2% by weight of a mixture of surfactants at the interface between the aqueous and lipid phases, the mixture of surfactants:
   comprising at least one amphiphilic lipid and at least one amphiphilic targeting ligand, and
   representing from 4 to 10% by weight, advantageously from 5 to 8% by weight based on the oil of the lipid phase;
      characterized in that the oil of the lipid phase comprises at least 70%, advantageously at least 80%, advantageously at least 95% by weight, notably at least 97% of C6-C18, advantageously C6-C14, more advantageously C6-C10 saturated fatty acid glycerides and in that the composition comprises more than 100 mmol, advantageously more than 120 mmol, even more advantageously more than 140 mmol of iron per liter of composition.

Advantageously, in the composition according to the invention, the total of the percentages of aqueous phase, of lipid phase and of surfactant at the interface of both of these phases is equal to 100%.

By the term of [symbol] fatty acid [symbol] are meant aliphatic carboxylic acids having a carbon chain with at least 4 carbon atoms. Natural fatty acids have a carbon chain from 4 to 28 carbon atoms (generally an even number). One refers to a [symbol] long chain fatty acid [symbol] for a length of 14 to 22 carbons and to a very long chain if there are more than 22 carbons. On the contrary one refers to a [symbol] short chain fatty acid [symbol] for a length from 6 to 10 carbons, in particular 8 or 10 carbon atoms. One skilled in the art is aware of the associated nomenclature and in particular uses:
Ci-Cp for referring to a range of Ci-Cp fatty acids
Ci+Cp, for the total of the Ci fatty acids and of the Cp fatty acids

For example:
fatty acids with 14 to 18 carbon atoms are written as [symbol] C14-C18 fatty acids [symbol]
the total of the C16 fatty acids and of the C18 fatty acids is written as C16+C18.
for an unsaturated fatty acid, one skilled in the art will use the following nomenclature Ci: x n−N wherein N will be the position of the double bond in the unsaturated fatty acid starting from the carbon opposite to the acid group, i is the number of carbon atoms of the fatty acid, x is the number of double bonds (unsaturations) of this fatty acid. Oleic acid will for example be designated by the nomenclature (C18:1n-9).

The iron concentration of the nanoemulsion composition is measured by techniques known to one skilled in the art and for example by atomic emission spectroscopy. In a preferential way, the nanoemulsion composition according to the invention comprises from 100 to 300 mmol of iron per liter of composition, more preferentially from 120 to 200 mmol of iron per liter of composition, even more preferentially from 140 to 160 mmol of iron per liter of composition.

Specificities on the various constituents of the composition are given hereafter.

### Aqueous Phase

The aqueous phase is advantageously water or a pharmaceutically acceptable aqueous solution such as a saline solution or a buffer solution. It may notably comprise certain additives such as glycerol or mannitol.

### Oil of the Lipid Phase

Very advantageously, the lipid phase is formed by oil and by magnetic particles (p).

Saturated fatty acid glycerides of the oil of the lipid phase are advantageously found in the form of saturated fatty acid triglycerides. The oil comprises at least 70%, preferably at least 80, 90, 95, 97% by weight of C6-C10 saturated fatty acid glycerides. This oil will have as advantages of being well suited for injectable pharmaceutical formulations of contrast agents, so as to not be sensitive to oxidation (thereby allowing the product comprising it to be kept for several months and the paramagnetic behavior of the product not being altered for medical imaging examinations) and especially to allow the composition of nanoemulsion for which the lipid phase comprises this oil or to the contrast product comprising this composition of having very good affinity towards the receptor which it targets.

Very advantageously, the oil comprises less than 10%, preferably less than 5% of glycerides of unsaturated fatty acids, in particular less than 5%, and preferably less than 2%, less than 1% by weight of C14-C18 or C14-C22 unsaturated fatty acid glycerides.

The oil of the lipid phase preferentially comprises or may consist of a mixture of diglycerides and/or triglycerides of one or more fatty acids selected from caprylic acid, capric acid, linoleic acid and succinic acid or one of their derivatives.

By derivatives of caprylic acid, of capric acid, of linoleic acid, of succinic acid, are meant methyl, hydroperoxyl, hydroxyl, oxoyl, epoxyl, methoxyl, halogenated, amine, cyanyl, nitrosyl or thiol derivatives of these various fatty acids.

Advantageously, the oil of the lipid phase comprises a mixture of caprylic acid and capric acid triglycerides.

Advantageously, the oil of the lipid phase comprises more than 80, 85, 90, 95% by weight of a mixture of caprylic acid and capric acid triglycerides.

For example, the oil of the lipid phase is copra oil or Miglyol® oil, notably of formula:

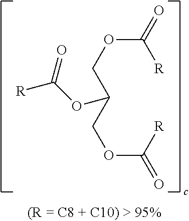

or one of its known derivatives, for example Miglyol® 810 oil, Miglyol® 812 oil (caprylic/capric triglyceride), Miglyol® 818 oil (caprylic/capric/linoleic triglyceride), Miglyol® 612 oil (glyceryl trihexanoate) or other derivatives Miglyol® propylene glycol dicaprylate dicaprate.

The copra oil has the following composition:

| Nature of the fatty acid of the fatty | |
|---|---|
| acid glycerides | Concentration (in % m/m) |
| Caprylic acid (C8:0) | 6-9% |
| Capric acid (C10:0) | 6-10% |
| Lauric acid (C12:0) | 44-51% |
| Myristic acid (C14:0) | 13-18% |
| Palmitic acid (C16:0) | 8-10% |
| Stearic acid (C18:0) | 1-3% |
| Oleic acid (C18:1 n-9) | 0.5-7.5% |
| Linoleic acid (C18:2 n-6) | <2.5% |

The oil of the lipid phase is preferentially a Miglyol® oil. For example the Miglyol® 812 oil has the following composition:

| Fatty acid of fatty acid | Concentration |
|---|---|
| glycerides | (m/m) |
| Caproic acid (C6:0) | max 2% |
| Caprylic acid (C8:0) | 50-65% |
| Capric acid (C10:0) | 30-45% |
| Lauric acid (C12:0) | Max 2% |
| Myristic acid (C14:0) | Max 1% |
| Linoleic acid (C18:2 n-6) | — |

The Miglyol® 818 oil itself has the following composition:

| Fatty acid of fatty acid | Concentration |
|---|---|
| glycerides | (m/m) |
| Caproic acid (C6:0) | max 2% |
| Caprylic acid (C8:0) | 45-65% |
| Capric acid (C10:0) | 30-45% |
| Lauric acid (C12:0) | Max 3% |
| Myristic acid (C14:0) | Max 1% |
| Linoleic acid (C18:2 n-6) | 2-5% |

According to alternatives, the oil of the lipid phase is a mixture of glycerides of saturated fatty acids comprising at least 70%, preferably at least 80, 90, 95% by weight of saturated fatty acid glycerides with 6 to 10 carbon atoms.

Preferably, the saturated fatty acid glycerides of the oil of the lipid phase are in the form of mono-, di- or tri-glycerides, preferably as triglycerides.

Preferably, the oil of the lipid phase of the emulsions comprises glycerides of saturated fatty acids, the saturated fatty acids of which are in the following alternatives:
C6-C18 >70% by weight, preferentially C6-C18 >80% by weight, more preferentially C6-C18 >95% by weight, and still more preferentially C6-C18 >98% by weight, or
C6-C14>70% by weight, preferentially C6-C14>80% by weight, more preferentially C6-C14>95% by weight, and even more preferentially C6-C14>98% by weight or
C8+C10>70% by weight, preferentially C8+C10>80% by weight, more preferentially C8+C10>95% by weight, and even more preferentially C8+C10>98% by weight or
C8 from 40 to 70% by weight, preferentially from 50 to 65% by weight and/or C10 from 20 to 50% by weight, more preferentially from 30 to 45% by weight, the total C8+C10 being greater than 80% by weight.

It was ascertained that beyond 49.5% by weight of the lipid phase in the composition, the latter adopts a too rheofluidifying behavior and/or an unsuitable viscosity (the viscosity becoming then greater than values from 4 to 5 mPa·s) for intravenous injection.

It is notably specified that taking into account the volume injectable to patients, of the order of 10 to 50 ml, the oil is used at a sufficiently high level, of at least 9.5% by weight based on the weight of the composition, in order to have a sufficiently concentrated solution both in droplets and in iron in order to obtain a sufficient MRI signal. It is necessary to have a concentration adapted to the injection duration, to the moment of acquisition of the signal and the associated processing of data by the practitioner. A too diluted solution would make it unusable for medical imaging examinations.

### Mixture of Surfactants at the Interface Between the Aqueous and Lipid Phases

It is recalled that the term of [symbol] tenside [symbol] or [symbol] surfactant [symbol] refers to a compound with an amphiphilic structure which gives it a particular affinity for interfaces of the oil/water or water/oil type which gives it the possibility of lowering the free energy of these interfaces and of stabilizing disperse systems.

One skilled in the art understands that the mixture of surfactants at the interface is represented by the whole of the surfactants used, i.e. as explained in detail in the application: amphiphilic lipids, amphiphilic targeting ligands, and if necessary other compounds such as pegylated lipids (lipids coupled with PEGs). Because of their amphiphilic structure, the amphiphilic targeting ligands play a role of a surfactant.

Examples of these compounds will be given in the following pages.

The nanodroplets each comprise a number of amphiphilic targeting ligands of the order of 100 to 5,000, notably 500 to 4,000, notably 1,800 to 3,500 (for example 2,000) which allows efficient targeting according to the affinity and the multivalence of the targeting ligand. The biological results obtained by means of the nanoemulsions according to the invention further show that the targeting ligands are advantageously distributed over the whole of the external surface of the nanodroplets, which is expressed by optimized multivalence of the targeting ligands.

Amphiphilic targeting ligands advantageously represent from 0.01 to 10% mole/mole of the total surfactant amount of the mixture of surfactants, more advantageously from 0.05 to 5%, notably from 0.05 to 3%.

By a [symbol] mole/mole [symbol] percentage (%) of the total amount of surfactant, is meant the number of amphiphilic targeting ligand moles for 100 moles of total surfactants of the mixture of surfactants.

The injected contrast product comprising the compositions of nanoemulsions described with an affinity advantageously of the order of 1 pM to 100 nM, notably 1 pM to 50 nM, advantageously 1 pM to 100 pM (the affinity per amphiphilic targeting ligand, around 1 nM to 1 μM is divided by the number of targeting ligands per nanodroplet).

Preferably, the mixture of surfactants at the interface between the aqueous and lipid phases of the nanoemulsion according to the invention, comprises from 80 to 96.95% mole/mole of amphiphilic lipid, from 3 to 15% mole/mole of pegylated lipid and from 0.05 to 5% mole/mole of an amphiphilic targeting ligand.

### Metal Particles (p)

By [symbol] particles based on an iron compound [symbol] , are meant in the sense of the present invention, particles comprising or consisting of an iron compound, generally comprising iron (III), generally an iron oxide or hydroxide.

As a general rule, the magnetic particles (p) consist entirely or partly of iron hydroxide; of iron oxide hydrate; of ferrites; of mixed iron oxides such as mixed iron oxides of cobalt, nickel, manganese, beryllium, magnesium, calcium, barium, strontium, copper, zinc or platinum; or a mixture thereof.

In the sense of the present application, the term of [symbol] ferrite [symbol] designates iron oxides of general formula [x Fe₂O₃, y MO_{z}], wherein M designates a magnetisable metal under the effect of a magnetic field such as Fe, Co, Ru, Mg, Mn, the magnetizable metal may optionally be radioactive.

Preferentially, the magnetic particles (p) of the compositions of the invention comprise a ferrite, notably maghemite (γ Fe₂O₃) or magnetite (Fe₃O₄), or further mixed cobalt ferrites (Fe₂CoO₄) or mixed manganese ferrites (Fe₂MnO₄). Within this context, most particularly magnetic particles (p) either totally or partly consisting of a ferrite are preferred, and essentially preferably (i.e. more than 90%, preferentially more than 95%, still more preferentially more than 98% by weight), of maghemite or magnetite or a mixture thereof.

The magnetic particles (p) are preferentially acid magnetic particles.

The magnetic particles (p) of the compositions according to the invention have at the surface protonated hydroxyl sites in an acid medium (i.e. a medium having a pH from 1 to 3.5, preferentially from 2 to 3) which more specifically corresponds to the species Fe—OH₂⁺ resulting from a very strong interaction between an Fe³⁺ ion at the surface of the particle and an acid water molecule (H₃O⁺). These protons may be considered as constituents of the particle according to the work of J Lyklema et al, Materials Science Research, 1984, 17, p. 1-24.

According to a more preferred alternative, the magnetic particles (p) are superparamagnetic particles.

The magnetic particles (p) before being covered with one or several fatty acids then preferably have a hydrodynamic diameter from 5 to 200 nm, still better from 5 to 60 nm or from 5 to 20 nm.

Very advantageously, the magnetic particles (p) based on an iron compound, are covered with an unsaturated, preferentially mono-unsaturated fatty acid, still more preferentially with oleic acid (C 18:1 n-9).

This fatty acid has the advantage of making the solubilization of the magnetic particles optimum in the oils of the lipid phase according to the invention.

### Other Features of the Nanoemulsions

The droplets of the nanoemulsions according to the invention have a sufficiently small size so as to allow them to circulate in biological media without degradation of the product, as far as the target of the amphiphilic targeting ligand inserted into the nanodroplets by means of its lipophilic group. The size of the nanodroplets is typically from 30 to 300 nm, advantageously 50 to 250 nm, notably 100 to 220 nm, in particular 180 to 210 nm. The size of the nanoemulsions is measured by using the PCS method (see details hereafter).

Further, the obtained formulations are iso-osmolar (i.e. their osmolarity is identical with that of the plasma), which avoids discomfort for the patient during the injection. Further, the amount of targeting ligands grafted to the nanodroplets is very well adapted for MRI imaging. The composition is further capable of supporting sterilization by heat, notably with an autoclave.

### Preferred Embodiments

According to preferred embodiments, the nanoemulsion composition according to the invention has as a weight composition:

1) from 50 to 90% by weight, advantageously from 60 to 85% by weight, more advantageously from 75 to 85% by weight, even more advantageously from 78 to 82% by weight of aqueous phase,

2) from 9.5 to 49.5% by weight, advantageously from 9.5 to 39.5% by weight, more advantageously from 14.5 to 24.5% by weight, even more advantageously from 17.5 to 21.5% by weight of lipid phase,

3) from 0.38 to 4.95% by weight of a mixture of surfactants (preferably from 4 to 10% of the lipid phase), preferentially from 0.5 to 2% by weight of a mixture of surfactants, the mixture of surfactants comprising from 90 to 99.99%, advantageously from 95 to 99.95%, more advantageously from 97 to 99.95% by mole/mole of amphiphilic lipid, and from 0.01 to 10% mole/mole, advantageously 0.05 to 5%, more advantageously 0.05 to 3% of amphiphilic targeting ligand, it being specified that the total of the percentages of 1), 2) and 3) is equal to 100%.

According to preferred embodiments, the nanoemulsion has the weight composition:

1) from 50 to 90% by weight, advantageously 60 to 85% by weight, more advantageously 75 to 85% by weight, still more advantageously 78 to 82% by weight of aqueous phase,

2) from 9.5 to 49.5% by weight, advantageously from 9.5 to 39.5% by weight, more advantageously from 14.5 to 24.5% by weight, still more advantageously from 17.5 to 21.5% by weight of lipid phase,

3) from 0.38 to 4.95% by weight of a mixture of surfactants, the mixture of surfactants comprising from 95 to 99.95% mole/mole of amphiphilic lipid and 0.05 to 5% mole/mole, notably 0.05 to 3% mole/mole of amphiphilic targeting ligand,

it being specified that the total of the percentages of 1), 2) and 3) is equal to 100%.

According to preferred embodiments, the nanoemulsion has the weight composition:

1) from 50 to 90%, preferably from 60 to 85%, advantageously from 75 to 85%, more advantageously 78 to 82%, notably 79 to 81% by weight of aqueous phase,

2) from 9.5 to 49.5%, preferably 9.5 to 39.5%, advantageously 14.5 to 24.5%, more advantageously 17.5 to 21.5% by weight of lipid phase comprising an oil, the oil comprising at least 70%, preferably at least 80, 90, 95% by weight of glycerides of saturated C6-C14 preferably C6-C10 fatty acids,

3) from 0.38 to 4.95%, advantageously 0.38 to 3.95%, more advantageously 0.5 to 1.5% by weight of a mixture of surfactants,

it being specified that the total of the percentages of 1), 2) and 3) is equal to 100%.

According to preferred embodiments, the mixture of the surfactants comprises (in mole/mole % of the total amount of surfactants):

3.1) from 50 to 99.99%, advantageously from 65 to 97.95%, more advantageously 77 to 97.95%, still more advantageously from 80 to 96.95% of amphiphilic lipid,

3.2) from 0.01 to 10%, advantageously from 0.05 to 5%, still more advantageously from 0.05 to 3% of amphiphilic targeting ligand,

3.3) from 0 to 40%, advantageously from 2 to 30%, more advantageously from 2 to 20%, still more advantageously from 3 to 15% of pegylated lipid, it being specified that the total of percentages of 3.1), 3.2) and 3.3) is equal to 100%.

In particular, the following embodiments are advantageous:

| | | % by weight of | % by weight of | |
|---|---|---|---|---|
| % by weight of | % by weight of | a mixture of | a mixture of | Iron |
| aqueous phase | lipid phase | surfactants | surfactants | concentration |
| based on the | based on the | based on the | based on the | (mmol/liter of |
| composition (1) | composition (2) | lipid phase (3) | composition (4) (*) | composition) |
| 50-90 | 9.5-40 | 4-10 of (2) | [0.38-4]% | 100-300 |
| 70-90 | 9.5-29.5 | 4-10 of (2) | [0.38-2.95]% | 100-300 |
| 75-85 | 14-25 | 4-10 of (2) | [0.56-2.5]% | 100-300 |
| 78-82 | 17-21 | 4-10 of (2) | [0.68-2.1]% | 100-300 |
| 75-85 | 14-25 | 5-8 of (2) | [0.7-2] % | 100-300 |
| 78-82 | 17-21 | 5-8 of (2) | [0.85-1.68]% | 100-300 |

It being specified that the total (1) + (2) + (4) = 100%
(*) the ranges of column 4 correspond to the percentages of column 3 multiplied by the percentages of column 2 (the smallest with the smallest and the greatest with the greatest).

For example, the range [0.38-4] corresponds to 4% (percentage specified in column 3)33 9.5 (percentage specified by column 2)=0.38% and 10% (percentage specified in column 3)×40 (percentage specified in column 2)=4%.

These ranges are notably preferred insofar that they give the possibility of obtaining a size of emulsion nanodroplets from 150 to 300 nm, and in particular around 180 to 210 nm.

The size and the stability of the emulsion nanodroplets are highly satisfactory, as well as the viscosity (of the order of 1 to 3 mPa.$). Their behavior is Newtonian, which is a significant advantage for injectable pharmaceutical solutions.

For example, one has the following ranges of proportions of the constituents.

| | | % by weight | | | Content % | |
|---|---|---|---|---|---|---|
| | | of a mixture | Content in % | | (mole/mole) | |
| | | of | (mole/mole) | Content % | of | |
| % by | | surfactants | of | (mole/mole) | amphiphilic | |
| weight of | % by | based on | amphiphilic | of pegylated | targeting | Iron |
| aqueous | weight of | the lipid | lipid in the | lipid in the | ligand in the | concentration |
| phase | lipid phase | phase | mixture of | mixture of | mixture of | (mmol/liter of |
| (1) | (2) | (3) | surfactants | surfactants | surfactants | composition) |
| 75 to 85, | 14 to 25, | 5 to 10, | 95 to 99.95 | 0 | 0.05 to 5, | 100 to 300, |
| preferably | preferably | preferably 5 | | | preferably | preferably 120 |
| 78 to 82, | 17 to 21, | to 8, | | | 0.05 to 3 | to 200, |
| preferably | preferably | preferably | | | | preferably 140 |
| 78.6 | 20 | 7.2 | | | | to 160 |
| 75 to 85, | 14 to 25, | 5 to 10, | 80 to 96.95 | 3 to 15, | 0.05 to 5, | 100 to 300, |
| preferably | preferably | preferably 5 | | preferably 5 | preferably | preferably 120 |
| 78 to 82, | 17 to 21, | to 8, | | to 15 | 0.05 to 3 | to 200, |
| preferably | preferably | preferably | | | | preferably 140 |
| 78.6 | 20 | 7.2 | | | | to 160 |
| 75 to 85, | 14 to 25, | 5 to 10, | 90 to 99.95 | 0 to 5 | 0.05 to 5, | 100 to 300, |
| preferably | preferably | preferably 5 | | | preferably | preferably 120 |
| 78 to 82, | 17 to 21, | to 8, | | | 0.05 to 3 | to 200, |
| preferably | preferably | preferably | | | | preferably 140 |
| 78.6 | 20 | 7.2 | | | | to 160 |

The total in the mixture of the surfactants of the contents of amphiphilic lipids, pegylated lipids, amphiphilic targeting ligands, being 100%.

### Amphiphilic Lipids

The amphiphilic lipids include a hydrophilic portion and a lipophilic portion. They are generally selected from compounds for which the lipophilic portion comprises a linear or branched, saturated or unsaturated, chain having from 8 to 30 carbon atoms.

They may be selected from phospholipids, cholesterols, lysolipids, sphingomyelins, tocopherols, glycolipids, stearylamines, cardiolipins of natural or synthetic origin; molecules consisting of a fatty acid coupled with a lipophilic group through an ether or ester function, such as sorbitan esters such as for example sorbitan mono-oleate and mono-laurate; polymerized lipids; sugar esters such as saccharose mono- and di-laurate, mono- and di-palmitate, mono- and distearate; said amphiphilic lipids may be used alone or as mixtures.

Advantageously, the amphiphilic lipid is a phospholipid, preferably selected from: phosphatidylcholine (also called lecithin), dioleoylphosphatidylcholine, dimyristoylphosphatidylcholine, dipalmitoylphosphatidylcholine, distearoylphosphatidylcholine, phosphatidylethanolamine, sphingomyelin, phosphatidylserine, phosphatidylinositol. Lecithin is a preferred amphiphilic lipid.

Advantageously, the amphiphilic lipid is a lipoid, notably EPC (Egg Phosphatidyl Choline and its derivatives notably known as Avanti Polar Lipids) or the lipoid® S75 of formula:

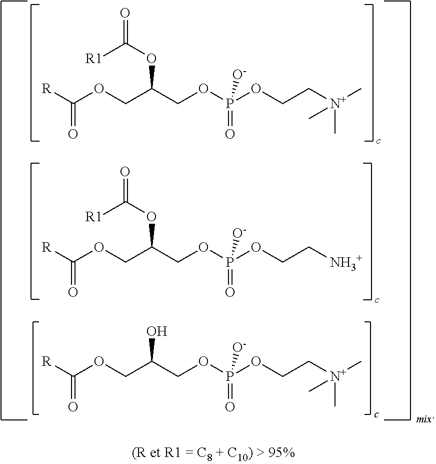

| | Proportions (m/m) |
|---|---|
| Phosphatidylcholine (+LPC) | 68 to 73% |
| Phosphatidylethanolamine | 7 to 10% |
| Lysophosphatidylcholine | <3% |
| Phosphorus | 3.4 to 3.7% |

According to a particular embodiment, all or part of the amphiphilic lipid may have a reactive function, such as a maleimide, thiol, amine, ester, oxyamine or aldehyde group. The presence of reactive functions allows grafting of functional compounds at the interface.

### Pegylated Lipids

It is possible to use for the lipid phase, in addition to the amphiphilic lipid and to the amphiphilic targeting ligand, in a non-mandatory way, and in particular in order to act on the stealth nature of the product in the body (i.e. giving the possibility of delaying the removal of the product by the reticular-endothelial system), pegylated lipids, i.e. bearing polyethylene oxide (PEG) groups such as polyethyleneglycol/phosphatidyl-ethanolamine (PEG-PE). By [symbol] polyethyleneglycol [symbol] PEG, in the sense of the present application, are generally designated compounds comprising a —CH₂—(CH₂—O—CH₂)ₖ—CH₂OR₃ chain wherein k is an integer varying from 2 to 100 (for example 2, 4, 6, 10, 50), and R₃ is selected from H, alkyl or —(CO)Alk, the term of [symbol] alkyl [symbol] or [symbol] Alk [symbol] designating here a linear or branched hydrocarbon aliphatic group having about 1 to 6 carbon atoms in the chain. The term of [symbol] polyethyleneglycol [symbol] as used here notably encompasses aminopolyethyleneglycol compounds. As an example of a pegylated lipid, mention may notably be made of PEG 350, 750, 2000, 3000, 5000, modified by adding amphiphilic groups in order to be inserted within the layer of surfactants of the nanodroplets, notably:

1,2-distearoyl-sn-glycero-3-phosphoethanolamine-N-[methoxy(polyethylene glycol)-350]
1,2-distearoyl-sn-glycero-3-phosphoethanolamine-N-[methoxy(polyethylene glycol)-550],
1,2-distearoyl-sn-glycero-3-phosphoethanolamine-N-[methoxy(polyethylene glycol)-750],

The pegylated lipid will notably be used:

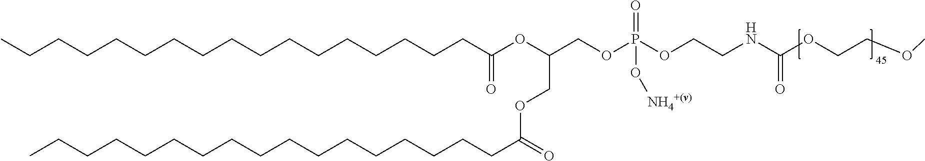

### Amphiphilic Targeting Ligands

The nanoemulsions are vectorized by means of amphiphilic targeting ligands. The nanoemulsion comprises at least one ligand for targeting a pathological area anchored to the nanodroplet, typically by means of the lipophilic group of the amphiphilic targeting ligand.

The nanoemulsions further have the advantage of being able to control the type and the amount of amphiphilic targeting ligands, and notably of being able to incorporate different amphiphilic targeting ligands. For example, a nanodroplet will comprise:
an amphiphilic targeting ligand which gives the possibility of access to a pathological physiological area, for example comprising a targeting ligand for passing through the BBB (blood brain barrier)
another amphiphilic targeting ligand which allows the targeting of a target biological marker over expressed by certain cells of this pathological area.

The molecular interaction between the targeting ligand of the amphiphilic targeting ligand and the target biological marker give the possibility of capturing nanodroplets at the pathological area, and the MRI imaging resulting from this allows specific localization of the pathological area.

Advantageously, the number of amphiphilic targeting ligands per droplet of nanoemulsion is of at least 50 and typically of the order of 500, 1,000, 2,000, 3,000, 5,000, 10,000.

The dosage of the amphiphilic targeting ligand may for example be carried out by MALDI-TOF mass spectrometry. The number of amphiphilic targeting ligands per nanoemulsion droplet is computed from measurement results of the hydrodynamic diameter (Z ave) obtained by PCS, of the oil volume in the emulsion and of the concentration of amphiphilic targeting ligands obtained by the method above.

As preferred targeting ligands of the amphiphilic targeting ligand, mention will be made of ligands of biological targets, the expression of which is modified in a pathological area (a tumor for example), relatively to the healthy area. Very preferentially, mention may be made as a biological target entering this definition, of integrins and notably the integrin αVβ3, which is the receptor for vitronectin and which in fact consists of two portions: the integrin alpha V and the integrin beta 3 (CD61). This integrin αᵥβ₃ is a target of choice for imaging in oncology since it is weakly expressed in healthy tissues but it is overexpressed during angiogenesis phenomena, like this is the case in most cancers, by the new vessels and in particular by the endothelial cells.

As a targeting ligand of the amphiphilic targeting ligand at least one targeting ligand is used, selected from: peptides (advantageously at least 20 amino acids, more advantageously from 5 to 10 amino acids), pseudopeptides, peptidomimetics, amino acids, agents for targeting integrins (peptides and pseudopeptides, peptidomimetics notably), glycoproteins, lectins, pteroic or aminopteroic derivatives, derivatives of folic and antifolic acid, antibodies or antibody fragments, steroids, oligonucleotides, sequences of ribonucleic acid, sequences of desoxyribonucleic acid, hormones, possibly recombinant or mutated proteins, mono- or poly-saccharides, compounds with a benzothiazole backbone, benzofurane, styrylbenzoxazole/thiazole/imidazole/quinoline, styrylpyridine and derived compounds, and mixtures thereof. Peptides, derivatives of folic and antifolic acid, agents for targeting integrins (peptides and pseudopeptides, peptidomimetics notably), agents for targeting cell receptors or enzymes (notably for targeting kinases, notably tyrosine kinase; metalloproteases; caspases . . . ) are most preferred.

[symbol] peptidomimetic [symbol] is meant to refer to a compound which does not consist of a regular sequence of amino acids connected together through peptide links but which mimics the biological activity of a peptide.

[symbol] pseudopeptide [symbol] is meant to refer to any peptide analog including at least one modification of the native peptide link —[CO—NH]—.

By [symbol] pteroic or aminopteroic derivatives [symbol] are meant compounds functionalized from pteroic or aminopteroic acid and/or modified by bioisostere groups as described in WO 2004/112839, WO 2007/042504, U.S. 2005/0227985 and WO 2010/102238.

By [symbol] derivatives of folic and antifolic acid [symbol] are meant compounds functionalized from folic acid or antifolic acid and/or modified by bioisostere groups as described in WO 2004/112839, WO 2007/042504, U.S. 2005/0227985 and WO 2010/102238. These derivatives notably include folinic acid, pteropolyglutamic acid and pteridine ligands of the folate receptor like tetrahydropterins, dihydrofolates, tetrahydrofolates and their deaza and dideaza analogs.

These [symbol] deaza [symbol] and [symbol] dideaza [symbol] analogs are analogs having a carbon atom substituted with one or two nitrogen atoms in the structure of folic acid or of antifolic acid. For example, the deaza analogs include the 1-deaza, 3-deaza, 5-deaza, 8-deaza, and 10-deaza analogs of the folate. The dideaza analogs for example include the 1,5-dideaza, 5,10-dideaza, 8,10-dideaza, and 5,8-dideaza analogs of the folate. As other derivatives of folic acid mention may also be made of aminopterin, amethopterin (methotrexate), N(10)-methylfolate, 2-deamino-hydroxyfolate, deaza analogs such as 1-deazamethopterin or 3-deazamethopterin and 3′,5′-dichloro-4-amino-4-deoxy-N(10)-methylpteroylglutamic acid (dichloromethotrexate).

By [symbol] compounds derived from styrylpyridine [symbol] , are meant compounds obtained from styrylpyridine and which bear at least a functionalized chain on one of the two aromatic rings of the latter.

According to advantageous embodiments, the targeting ligand (i.e. the portion allowing the targeting) of the amphiphilic targeting ligand is selected from the following list (the documents and references between brackets are examples and not a limiting list):
1) Targeting ligands targeting VEGF receptors and angiopoietin (described in WO 01/97850), polymers such as polyhistidine (U.S. Pat. No. 6,372,194), polypeptides targeting fibrin (WO 01/09188), peptides targeting integrins (WO 01/77145, WO 02/26776 for alphaV-beta3, WO 02/081497, for example RGDWXE), pseudopeptides and peptides for targeting metalloproteases MMP (WO 03/062198, WO 01/60416), peptides for example targeting the receptor KDR/Flk-I including R-X-K-X-H and R-X-K-X-H, or receptors Tie-1 and 2 (WO 99/40947 for example), Lewis sialyl glycosides (WO 02/062810 and “Müller et al, Eur. J. Org. Chem, 2002”, 3966-3973), antioxidants such as ascorbic acid (WO 02/40060), ligands for targeting tuftsin (for example U.S. Pat. No. 6,524,554), targeting receptors of G protein GPCR in particular cholecystokinin (WO 02/094873), associations between integrin antagonist and mimetic of guanidine (U.S. Pat. No. 6,489,333), quinolones targeting alphaVbeta3 or 5 (U.S. Pat. No. 6,511,648), benzodiazepines and the like targeting integrins (U.S. 2002/0106325, WO 01/97861), imidazoles and the like (WO 01/98294), RGD peptides (WO 01/10450), antibodies or antibody fragments (FGF, TGFb, GV39, GV97, ELAM, VCAM, which may be induced by TNF or IL (U.S. Pat. No. 6,261,535), targeting molecules modified by interaction with the target (U.S. Pat. No. 5,707,605), agents for targeting amyloid deposits (WO 02/28441 for example), cathepsin-cleaved peptides (WO 02/056670), mitoxantrone or quinone (U.S. Pat. No. 6,410,695), polypeptides targeting epithelial cells (U.S. Pat. No. 6,391,280), inhibitors of cystein proteases (WO 99/54317), targeting ligands as described in: U.S. Pat. No. 6,491,893 (GCSF), U.S. 2002/0128553, WO 02/054088, WO 02/32292, WO 02/38546, WO 03/006059, U.S. Pat. No. 6,534,038, WO 01/77102, EP 1 121 377, Pharmacological Reviews (52, no. 2, 179; growth factors PDGF, EGF, FGF . . . ), Topics in Current Chemistry (222, W. Krause, Springer), Bioorganic & Medicinal Chemistry (11, 2003, 1319-1341; tetrahydrobenzazepinones derivatives targeting alphaV-beta3).
2) Angiogenesis inhibitors, notably those tested in clinical trials or already marketed, notably:
angiogenesis inhibitors involving FGFR or VEGFR receptors such as SU101, SU5416, SU6668, ZD4190, PTK787, ZK225846, azacycle compounds (WO 02/44156, WO 02/059110);
angiogenesis inhibitors involving MMPs such as BB25-16 (marimastat), AG3340 (prinomastat), solimastat, BAY12-9566, BMS275291, metastat, neovastat;
angiogenesis inhibitors involving integrins such as SM256, SG545, adhesion molecules blocking EC-ECM (such as EMD 121-974, or vitaxin);
drugs with a more indirect anti-angiogenic action mechanism such as carboxyamidotriazole, TNP470, squalamine, ZD0101;
the inhibitors described in document WO 99/40947, the monoclonal antibodies which are very selective for binding to the KDR receptor, the analogs of somatostatin (WO 94/00489), the peptides for binding to selectin (WO 94/05269), growth factors (VEGF, EGF, PDGF, TNF, MCSF, interleukins); targeting ligands for targeting VEGF as described in Nuclear Medicine Communications, 1999, 20;
the inhibitor peptides of document WO 02/066512.
3) Targeting ligands capable of targeting the receptors: CD36, EPAS-1, ARNT, NHE3, Tie-1, 1/KDR, Flt-1, Tek, neuropilin-1, endoglin, pleientropin, endosialin, Axl., alPi, a2ssl, a4P1, a5pl, eph B4 (ephrin), laminin A receptor, neutrophilin 65 receptor, leptin OB-RP receptor, chemokine CXCR-4 receptor (and other receptors cited in document WO99/40947), LHRH, bombesin/GRP, gastrin receptors, VIP, CCK.
4) Targeting ligands of the tyrosine kinase inhibitor type.
5) Known inhibitors of the GPIIb/IIIa receptor selected from: (1) the fab fragments of a monoclonal antibody of the GPIIb/IIIa receptor, Abciximab, (2) small peptide or peptidomimetic molecules injected intravenously such as eptifibatid and tirofiban.
6) Antagonist peptides of fibrinogen receptors (EP 0 425 212), ligand peptides of IIb/IIIa receptors, ligands of fibrinogen, ligands of thrombin, peptides capable of targeting atheromatous plaque, platelets, fibrin, peptides based on hirudin, derivatives based on guanine targeting the IIb/IIIa receptor.
7) Other targeting ligands or biologically active fragments of targeting ligands known to one skilled in the art as drugs, with an anti-thrombotic action, antiplatelet aggregation, antiatherosclerotic, antirestenotic, anticoagulant action.
8) Other targeting ligands or biologically active fragments of targeting ligands targeting alpha V beta3, described in association with DOTAs in patent U.S. Pat. No. 6,537,520, selected from the following substances: mitomycin, tretinoin, ribomustin, gemcitabin, vincristin, etoposide, cladribin, mitobronitol, methotrexate, doxorubicin, carboquone, pentostatin, nitracrin, zinostatin, cetrorelix, letrozole, raltitrexed, daunorubicin, fadrozole, fotemustin, thymalfasin, sobuzoxane, nedaplatin, cytarabin, bicalutamide, vinorelbin, vesnarinone, aminoglutethimide, amsacrin, proglumide, elliptinium acetate, ketanserin, doxifluridin, etretinate, isotretinoin, streptozocin, nimustin, vindesin, flutamide, drogenil, butocin, carmofur, razoxane, sizofilan, carboplatin, mitolactol, tegafur, ifosfamide, prednimustin, picibanil, levamisole, teniposide, improsulfan, enocitabin, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, luteinizing hormone releasing factor.
9) Certain targeting ligands targeting particular types of cancers, for example peptides targeting the ST receptor associated with colorectal cancer, or the tachykinin receptor.
10) Targeting ligands using compounds of the phosphine type.
11) Targeting ligands for targeting P-selectin, E-selectin; for example, the peptide with 8 amino acids described by Morikawa et al, 1996, 951, as well as various sugars.
12) Annexin V or targeting ligands targeting apoptotic processes.
13) Any peptide obtained by targeting technologies such as phage display, optionally modified with non-natural amino acids (http//chemlibrary.bri.nrc.ca), for example peptides from phage display banks: RGD, NGR, CRRETAWAC, KGD, RGD-4C, XXXY*XXX, RPLPP, APPLPPR.
14) Other peptide targeting ligands known for targeting atheromatous plaques, notably mentioned in document WO 2003/014145 and notably VCAM
15) Vitamins.
16) Ligands of hormonal receptors including hormones and steroids.
17) Targeting ligands targeting opioid receptors.
18) Targeting ligands targeting TKI receptors.
19) LB4 and VnR antagonists.
20) Nitroimidazole and benzylguanidine compounds.
21) Targeting ligands recalled in Topics in Current Chemistry, Vol. 222, 260-274, Fundamentals of Receptor-based Diagnostic Metallopharmaceuticals, notably:
targeting ligands for targeting peptide receptors overexpressed in tumors (LHRH receptors, bombesin/GRP, VIP receptors, CCK receptors, tachykinin receptors, for example), notably the analogs of somatostatin or bombesin, derived octreotide peptides optionally glycosylated, VIP peptides, alpha-MSH, CCK-B peptides.
peptides selected from: RGD cyclic peptides, fibrin-alpha chain, CSVTCR, tuftsin, fMLF, YIGSR (receptor: laminin).
22) Oligosaccharides, polysaccharides and ose derivatives, derivatives targeting GLUT receptors (ose receptors).
23) Targeting ligands used for products of the smart type.
24) Markers of myocardial viability (tetrofosmin and hexakis-2-methoxy-2-methylpropylisonitrile).
25) Tracers of the metabolism of sugars and fats.
26) Ligands of neurotransmitter receptors (D, 5HT, Ach, GABA, NA receptors).
27) Oligonucleotides.
28) Tissue factor.
29) Targeting ligands described in WO 03/20701, in particular the PK11195 ligand of the peripheral benzodiazepin receptor.
30) Peptides binding fibrin, notably the peptide sequences described in WO 03/11115.
31) Inhibitors of aggregation of amyloid plaques (for example described in WO 02/085903).
32) Pharmacophores, compounds for targeting Alzheimer's disease, in particular the compounds comprising backbones of the benzothiazole, benzofurane, styrylbenzoxazole/thiazole/imidazole/quinoline, styrylpyridin type.
33) Pharmacophores, compounds for targeting obtained from chemical backbones with pharmacological activity as described in U.S. 2007098631, notably the formulae of pages 4 to 10 and pages 13-14 (incorporated by reference), notably the compounds of the table on page 4 in the column entitled [symbol] scaffolds and derivatives [symbol] : biphenyl; arylpiperidine; arylpiperazine; 1,4-dihydropyridine dihydropyrimidone; 1,4-benzodiazepin-2-one; 1,5-benzodiazepin-2-one; 1,4-benzodiazepin-2,5-diones; pyrrolo-2,1-c-1,4-benzodiazepin-5,11-diones; 1,4-benzothiazepin-5-ones; 5,11-dihydro-benzopyrido-3,2b-1,4-diazepin-6-ones, benzopyrane; chromone; benzopyranone; coumarin, pyranocoumarin; benzopiperazinones; quinazolinone; quinazolindione; quinoxalinone; imidazoquinoxaline; indole; benzimidazole, benzofurane, benzothiophene.
34) Ligands for targeting integrins, notably mimetic non-peptide compounds of RGD peptides, and in particular tetrahydronaphthyridine compounds for example described in: J Med. Chem., 2003, 46, 4790-4798, Bioorg. Med. Chem. Letters, 2004, 14, 4515-4518, Bioorg. Med. Chem. Letters, 2005, 15, 1647-1650.
35) Ligands for targeting MUC5AC, notably antibody fragments, peptides and mimetic non-peptide compounds of peptides.
36) Ligands of a target associated with angiogenesis, notably a target selected from 4N1 K, AGF (angiopoietin-related growth factor), Angiogenin (ANG), Angiopoietin-1, Angiopoietin-2, Angiostatin, ARP4 (angiopoietin-related protein 4), bFGF (basic fibroblast growth factor), CD31 (PECAM-1), CD34, CD97, CD146 (MUC18), CECs (circulating endothelial cells), CEPs (circulating endothelial precursor cells), Collagenase-1 (C1), COX-2 (Cyclooxygenase-2), E7820, EG-1 (endothelial-derived gene-1), Extra-Domaine B (ED-B) of Fibronectin, Endoglin (CD105), ESAF (Endothelial cell stimulating angiogenesis factor), Factor VIII (anti-hemophilic factor A), Flt-1 (Fms-like tyrosine kinase 1), Integrin α1α2, Integrin α2β1, Integrin α3β1, Integrin α5β1, Integrin αvβ3, Integrin α6β4, Integrin α9β1, Integrin-β1, KDR, N-Cadherin, Nestin, NG2 proteoglycan, PSMA (prostate-specific membrane antigen), PV-1 (Plasmalemma vesicle associated protein-1), S100A13, Syndecane-1, T-Cadherin, TEM-5 (Tumor endothelial marker 5), TEM-8 (Tumor endothelial marker-8), Thrombospondin-1 (TSP1), Thrombospondin-2 (TSP2), Thy-1, Tie-1, Tie-2, Tn-C (Tenascin-C), TP (Thymidine phosphorylase), VCAM-1 (vascular cell adhesion molecule-1), VE-cadherin, VEGF, VWF (von Willebrand Factor) and the inhibitors of angiogenesis (recalled in paragraph 2) above.

In particular, for these tetrahydronaphthyridine compounds, any naphthyridine compound known from the prior art may be used (notably those of WO 2009/114776), the use of naphthyridine compounds as a targeting ligand for medical imaging being described in WO 2007/042506 page 13, lines 30-34.

The targeting ligand of the preferential amphiphilic targeting ligand according to the invention is selected from peptides and/or peptidomimetics and/or ligands for targeting integrins (for example the integrin αVβ3). Advantageously, the targeting ligand of the amphiphilic targeting ligand is a peptidomimetic. Very advantageously, the targeting ligand of the amphiphilic targeting ligand is a peptidomimetic compound for targeting integrins and in particular, a peptidomimetic compound for targeting integrins selected from compounds comprising a naphthyridine group. Even more advantageously, the amphiphilic targeting ligand is a peptidomimetic compound for targeting integrins of formula:

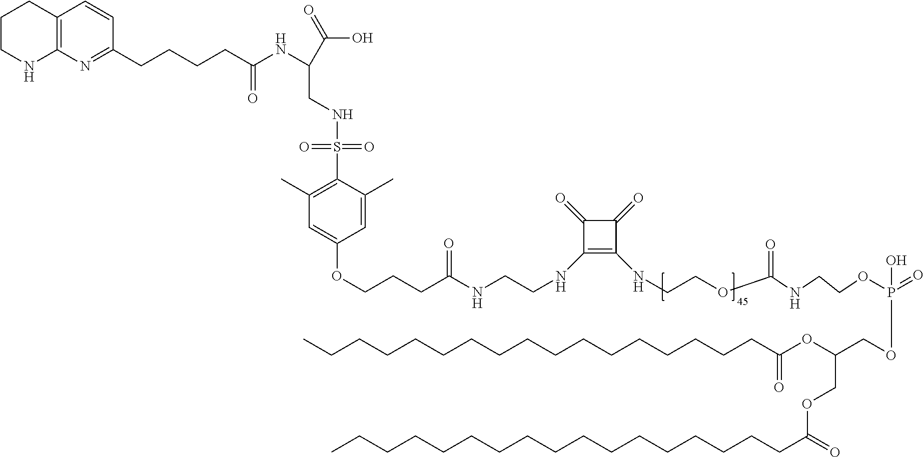

or one of its salts, for example as an ammonium phosphate.

This peptidomimetic of RGD has the property of targeting the integrin αVβ3.

The targeting ligands of the amphiphilic targeting ligand for recognizing the target in a biological medium are essentially located on the side of the external surface of the nanodroplets, the lipophilic group of the amphiphilic targeting ligand being inserted into the surfactant layer.

The amphiphilic targeting ligand is advantageously written as: Bio-L-Lipo wherein:
Bio is the targeting ligand (notably one of the targeting ligands mentioned above) localized at the external surface of the nanodroplets,
Lipo is a lipophilic group being inserted within the surfactant layer,
L is a binding group connecting Bio and Lipo, advantageously selected from:
   a single bond, squarate, C1-C6 alkylene, PEG, for example CH₂—(CH₂—O—CH₂)ₖ—CH₂ with k=1 to 10, (CH₂)₃—NH, NH—(CH₂)₂—NH, NH—(CH₂)₃—NH, (CH₂)ₙ, (CH₂)ₙ—CO—, —(CH₂)ₙNH—CO— with n=2 to 10, (CH₂CH₂O)_{q}(CH₂)ᵣ—CO—, (CH₂CH₂O)_{q}(CH₂)ᵣ—NH—CO— with q=1-10 and r=2-10, (CH₂)ₙ—CONH—, (CH₂)ₙ—CONH-PEG, (CH₂)ₙ—NH—, —OOC—CH₂—O—(CH₂)₂—O—(CH₂)₂—O—CH₂—COO—; —OOC—(CH₂)₂—CO₂—(CH₂)₂—OCO—(CH₂)₂—COO—; —OOC—CH(OH)—CH(OH)—COO—; —OOC—(CH₂)ₙ—COO—; —NH—(CH₂)ₙ—NH—, with n=0-20; NH—(CH₂)ₙ—CO₂; NH—CH₂—(CH₂—O—CH₂)ₙ—CO₂ with n=1 to 10,
   P1-I-P2, either identical or different, P1 and P2 being independently selected from O, S, NH, a simple bond, CO₂, NHCO, CONH, NHCONH, NHCSNH, SO₂NH—, NHSO₂—, squarate
      with I=alkylene, alkoxyalkylene, polyalkoxyalkylene (notably PEG), an alkylene interrupted with one or several squarates, with one or several aryls, advantageously phenyls, or with one or several groups selected from —NH—, —O—, —CO—, —NH(CO)—, —(CO)NH—, —O(CO)—, and —(OC)O—, alkenylene or alkynylene, said alkenylene or alkynylene being optionally interrupted with one or several groups selected from —NH—, —O—, —CO—, —NH(CO)—, —(CO)NH—, —O(CO)—, or —(OC)O—.

The covalent bonds L or I between Bio and Lipo are advantageously of the type —CONH—, —COO—, —NHCO—, —OCO—, —NH—CS—NH—, —C—S—, —N—NH—CO—, —CO—NH—N—, —CH₂—NH—, —N—CH₂—, —N—CS—N—, —CO—CH₂—S—, —N—CO—CH₂—S—, —N—CO—CH₂—CH₂—S—, —CH═NH—NH—, —NH—NH═CH—, —CH═N—O—, —O—N═CH— or fitting the following formulae:

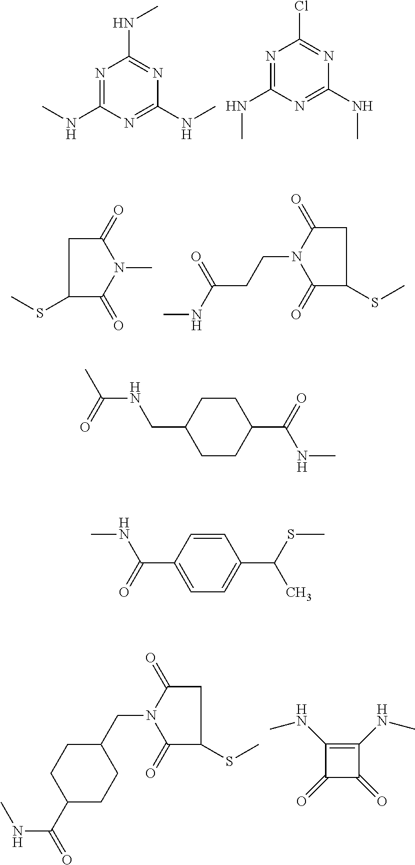

When the divalent groups mentioned above as possible groups L or I are not symmetrical, it is understood that the group Bio- or Bio-P1- may be grafted on the right or on the left of said covalent group. For example, when L is written, it represents a group —COO—, the targeting ligand may be Bio-COO-Lipo or Lipo-COO-Bio.

A few examples of amphiphilic targeting ligands are shown (hereafter: peptides, derivatives of folic acid, naphthyridine derivatives), made to be amphiphilic for anchoring to the external surface of the nanodroplet.

The application shows illustrative and non-limiting examples of their synthesis.

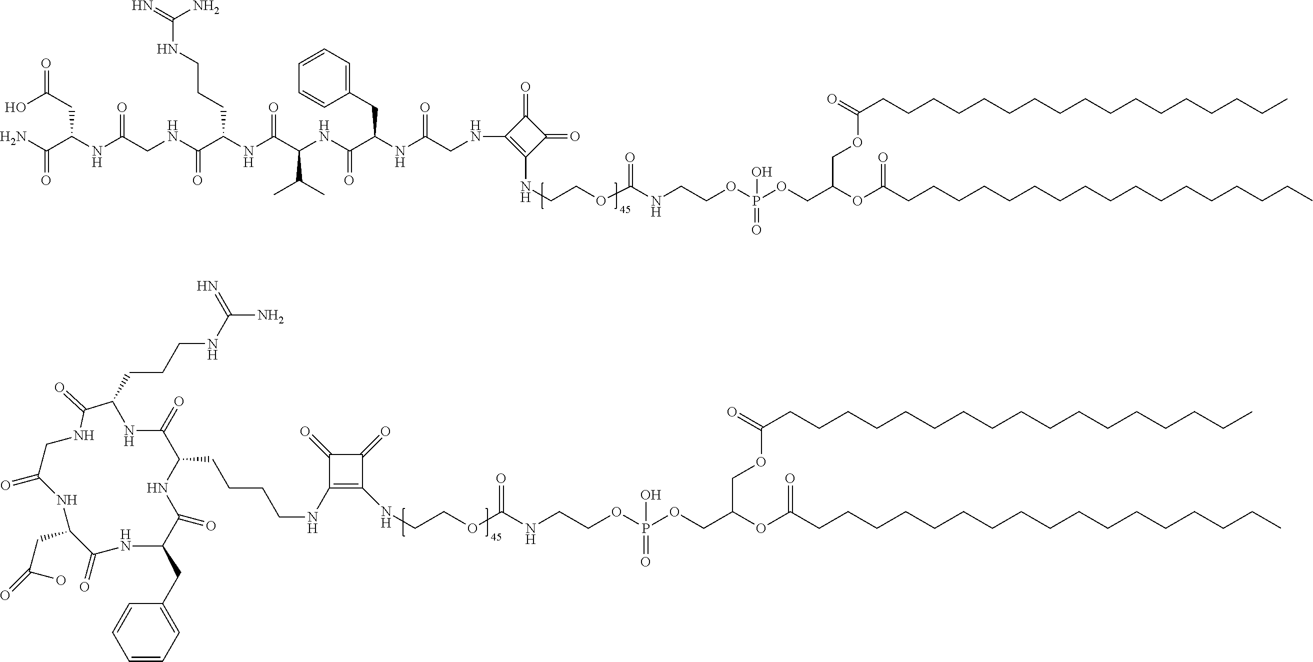

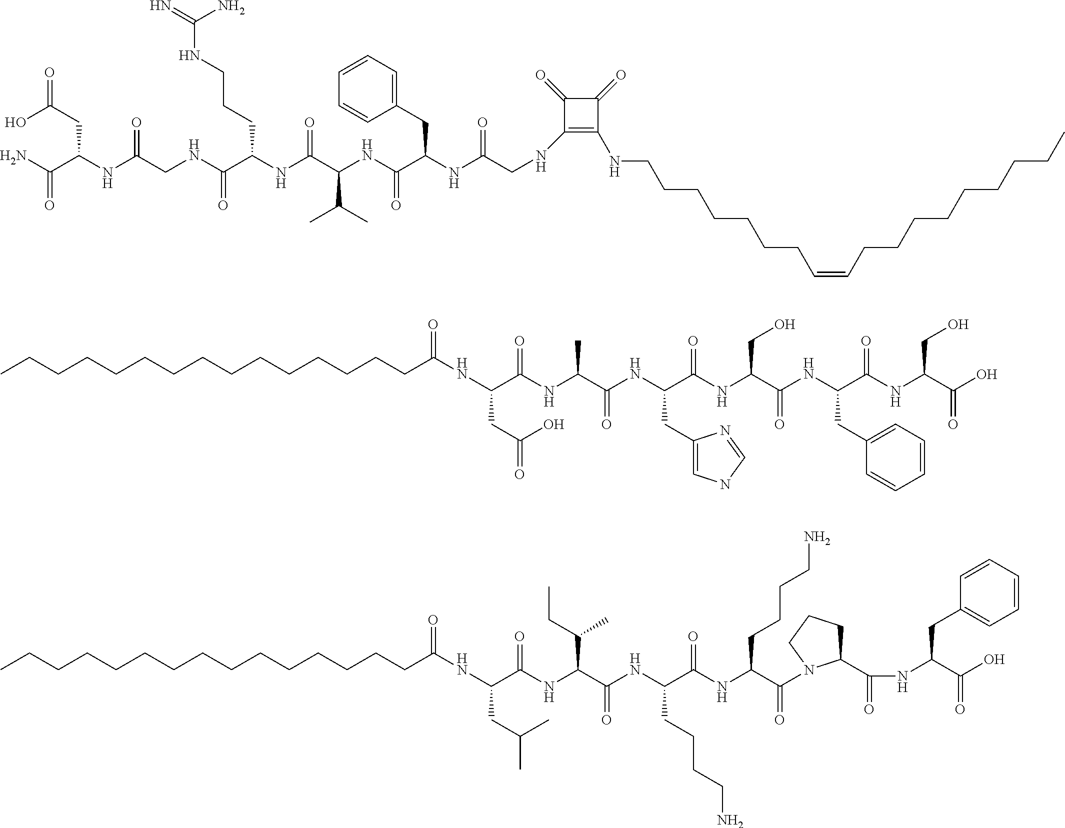

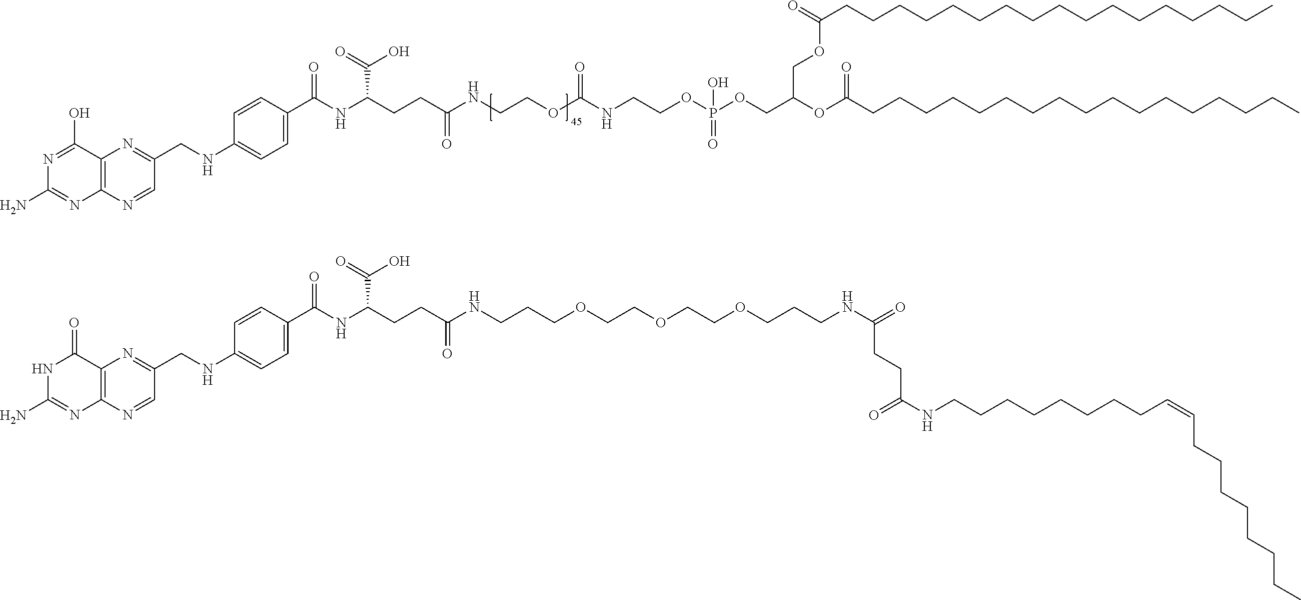

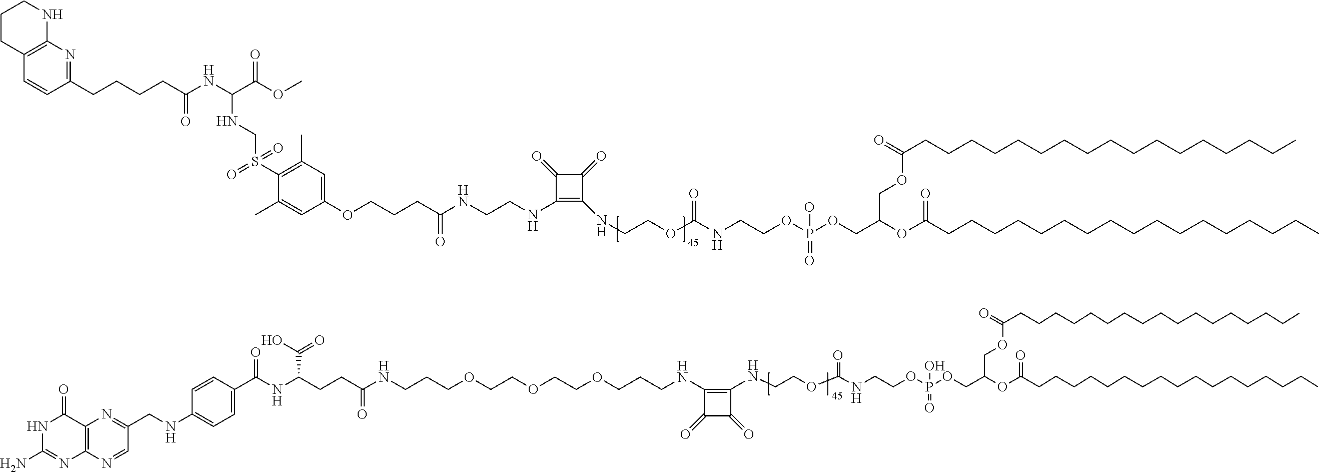

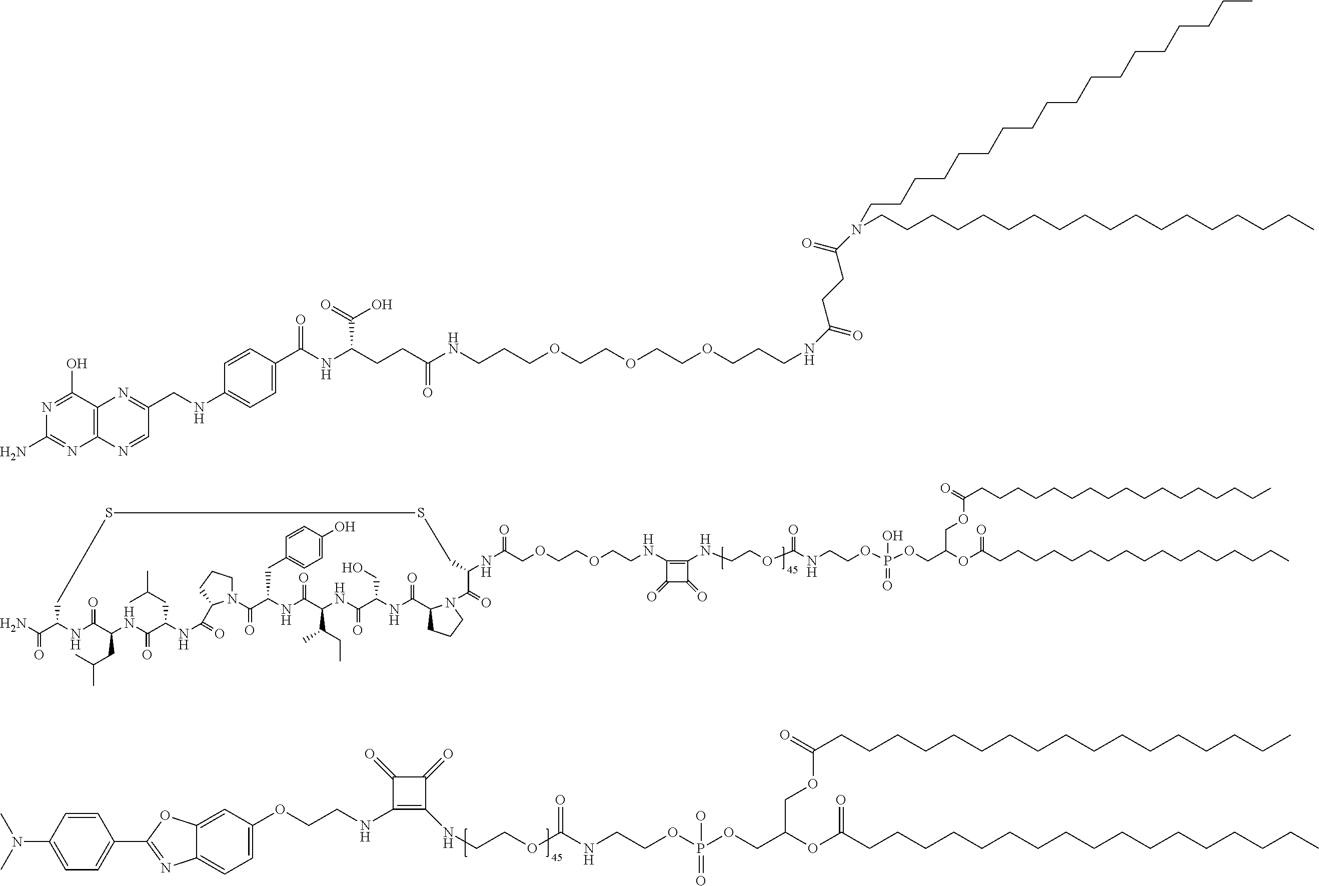

As explained in the application, the compositions are essentially used for diagnostic imaging. However, it is possible to prepare nanoemulsions further comprising ligands comprising a therapeutic agent. The nanodroplets will then comprise an amphiphilic targeting ligand in order to attain the biological target (the pathological area) on the one hand, and a ligand comprising a therapeutic agent for the therapeutic treatment on the other hand. The invention thus also relates to the compositions described previously, comprising a therapeutic agent, for their use for the treatment of diseases, notably cancer, neurodegenerative, vascular diseases.

According to embodiments, the mixture of surfactants further comprises at least one amphiphilic stealth agent, advantageously a PEG derivative, a ganglioside derivative (oside residues typically esterified by sialic acid or NAC), a polysaccharide (notably dextran or one of its known derivatives). These stealth agents are integrated without altering the affinity of the nanodroplet for the biological target.

### Method for Preparing a Nanoemulsion According to the Invention

According to another aspect, the invention relates to a method for preparing a composition as defined above, comprising the steps of:

a) Solubilizing magnetic particles (p) based on an iron compound, covered with one or several C8-C22 fatty acids, in an oil comprising at least 70%, preferably at least 80, 90, 95, 97% by weight of glycerides of C6-C18, advantageously C6-C14 and very advantageously C6-C10 saturated fatty acids in order to form a lipid phase,

b) Mixing the lipid phase obtained in step a) and an aqueous phase comprising the mixture of the surfactants, so as to form nanodroplets,

c) recovering the obtained nanoemulsion.

Preferably, the solubilization of step a) is total. By [symbol] total solubilization [symbol] is meant that after mixing the oil and the magnetic particles (p) based on an iron compound, covered beforehand with one or several C8-C22 fatty acids, there is an absence of aggregate visible to the naked eye in the formed lipid phase.

The magnetic particles (p) based on an iron compound are covered with one or more C8-C22 fatty acids, for example by diluting the magnetic particles in an alkaline solution (for example a 5.10⁻³ M soda (NaOH) solution) and adding a large excess (50 to 300, preferentially 50 to 150 equivalents) of one or more C8-C22 fatty acids, as described above, leading to precipitation of the magnetic particles.

The step a) for solubilizing magnetic particles (p) in the oil is generally carried out by stirring at a temperature above 60° C., preferentially above 80° C., even more preferentially above 90° C., or even above 100° C.

The stirring during the solubilization step a) may be carried out for 10 to 40 hours, preferentially 15 to 30 hours, still more preferentially 24 hours.

The mixture of surfactants (amphiphilic lipid, amphiphilic targeting ligand and pegylated lipid if required) is generally added into the aqueous phase (defined above) by dispersion, for example by means of ultrasonic waves, in other words by sonication.

During step b), the nanoemulsion is advantageously obtained by emulsification in a microfluidizer. The thereby obtained nanoemulsion is then used for administration to the patient, if required after incorporating various pharmaceutical additives. It may also be filtered. The obtained nanoemulsions may be freeze-dried with, if required, the use of anti-agglutination agents.

The following characteristics are typically obtained (obtained by means of analytical methods specified at the beginning of the example part hereafter), which may vary depending on the specific compositions of the emulsions and on their preparation method:
hydrodynamic diameter (Z ave): 150 to 220 nm, preferentially 185 to 200 nm, even more preferentially 190 to 200 nm.
polydispersity index (Poly a): 0.01 to 0.1, preferentially 0.03 to 0.08, still more preferentially 0.04 to 0.06.
iron concentration (mmol/liter of composition): 100 to 300 preferentially 120 to 200, still more preferentially 140 to 160.
droplet concentration in the nanoemulsion: 80 to 200 nM, preferentially 90 to 120 nM.
relaxivity at 60 MHz (water medium/37° C.)
   r1 (mM⁻¹ (Fe) s⁻¹): 0.5 to 4, preferably 0.8 to 2, even more preferentially 1.
   r2 (mM⁻¹ (Fe) s⁻¹): 180 to 250, preferentially 190 to 220, still more preferentially 210.
osmolality: 280-320 mOsm/kg.
stability in a pharmaceutical solution: for more than 6 months.
stability in Seronorm™ (biological stability): for more than 24 h.
number of magnetic particles per nanoemulsion droplet: more than 90, preferentially more than 100, still more preferentially more than 140.
number of amphiphilic targeting ligands per nanoemulsion droplet: 100 to 5,000, notably 500 to 4,000, advantageously 1,800 to 3,500, still more advantageously 2,500 to 3,500.

Globally, the nanoemulsions obtained for MRI:
have greater efficiency than those of the emulsions presently known in terms of signal for clinical imaging (MRI in particular) in patients,
have an affinity towards receptors which they target, greater than that of other emulsions non-compliant with the present invention,
are capable of incorporating ligands for targeting pathological areas at the surface of the nanodroplets, in a suitable amount and without any bothersome loss of affinity with their biological target,
are sufficiently stable chemically so as to be produced and preserved for a long time (several months to several years), in particular without any coalescence problem of the droplets with each other,
are sufficiently stable in vivo so as not to be degraded,
are adapted as regards pharmacokinetics,
are produced with oils listed in the pharmacopeia.

### Contrast Product Comprising the Nanoemulsion Composition According to the Invention

According to another aspect, the invention relates to a contrast product, preferably for MRI, comprising the nanoemulsion composition as described above.

The contrast product is preferably administered via an intravascular route, depending on the examined patient, for example, for a composition having an iron concentration of 145 mM, in an amount of 50 to 180, preferentially 80 to 120, highly preferentially 90 to 110, even more preferentially 100 pmoles of iron per kg of patient, which corresponds to volumes of 0.34 to 1.24, preferentially 0.55 to 0.83, even more preferentially 0.7 ml of contrast product per kg of patient.

The contrast product may be formulated by means of known additives for example recalled in U.S. Pat. No. 6,010,682, notably for administration via an intravenous injection. It may notably comprise thickeners, saccharides or polysaccharides, glycerol, dextrose, sodium chloride and/or antimicrobial agents.

### Use of the Nanoemulsion Compositions and Contrast Products According to the Invention

The invention also relates to the compositions described earlier for their use for diagnosing diseases, notably cancer, inflammatory, neurodegenerative or vascular diseases, notably cardiovascular diseases.

The invention relates to a method for imaging the entire body or a portion of the body of an individual comprising a step for obtaining one or more images of the entire body or of a portion of the body of an individual by a medical imaging technique, wherein said entire body or said portion of the body of the individual comprises the composition defined above or the contrast product defined above (preferably in an effective amount) and in which said image(s) are associated with magnetic particles based on an iron compound contained in the composition defined above or in the contrast product defined above.

According to an embodiment, the imaging method according to the invention does not include any invasive administration or injection step for the composition or the contrast product for the individual.

According to another embodiment, the imaging method according to the invention comprises a preliminary step for injecting or administering the composition or the contrast product to the individual, preferably an injection via an intravascular route.

This method allows dynamic acquisition by viewing the enhancement phases over time of the area of interest and of the wash-out area.

The clinical data obtained may give the possibility of helping a physician to determine whether the patient should receive or not a given therapeutic treatment, for example an anti-angiogenic treatment.

Thus, the invention also relates to a method for evaluating angiogenesis in an individual comprising the steps consisting of:

a) obtaining one or more images of the entire body or of a portion of the body of an individual by a medical imaging technique, wherein the entire body or said portion of the body of the individual comprises:
the composition for which the targeting ligand of the amphiphilic targeting ligand is a ligand of a target associated with angiogenesis as defined above, or
the contrast product comprising a composition for which the targeting ligand of the amphiphilic targeting ligand is a ligand of a target associated with angiogenesis as defined above
   and wherein said image(s) is(are) associated with magnetic particles based on an iron compound contained in said composition or in said contrast product;

b) evaluating angiogenesis in the entire body or in the portion of the body of the individual from said image(s) obtained in step a).

The imaging method defined above may also allow evaluation of the effectiveness of a treatment, notably of an anti-angiogenic treatment, preferentially an anti-cancer treatment and more preferentially an anti-angiogenic anti-cancer treatment (of the Avastin® type for example).

Thus, the invention also relates to a method for evaluating the effectiveness of an anti-angiogenic treatment comprising the steps consisting of:
a) evaluating angiogenesis in an individual having received an anti-angiogenic treatment, by the method defined above;
b) comparing the angiogenesis evaluated according to step a) with an angiogenesis reference in the individual before the anti-angiogenic treatment.

The reference may be the level existing before any anti-angiogenic treatment in the individual (base level) or any previous step for therapeutic treatment.

The anti-angiogenic treatment is considered as effective if reduction or maintaining of angiogenesis is observed relatively to the reference, ineffective if an increase in angiogenesis is observed relatively to the reference.

The method, according to an embodiment, does not include any step for administration of the anti-angiogenic treatment.

In an embodiment, the method for evaluating the effectiveness of an anti-angiogenic treatment defined above comprises:

a) evaluating angiogenesis in an individual in order to determine the base level by the method defined above,

b) administering an anti-angiogenic treatment to said individual, preferably by an injection via an intravascular route,

c) evaluating angiogenesis after said treatment in the individual in order to determine a level after treatment by the method defined above,

d) comparing the level after treatment and the base level.

In the methods defined above, the images are preferably obtained by Magnetic Resonance Imaging (or MRI) or Magnetic Particle Imaging (MPI).

By [symbol] effective amount [symbol] , is meant an amount of a composition of nanoemulsions or of a contrast product comprising this nanoemulsion composition, which gives the possibility of obtaining images by the medical imaging technique used.

The examples appearing hereafter are shown as an illustration and not as a limitation of the invention.

### DESCRIPTION OF THE FIGURES

FIG. 1: diagram of the structure of an exemplary nanoemulsion according to the invention.

1 designates a magnetic particle (p) based on an iron compound and covered with one or more C8-C22 fatty acids.

2 designates the lipid phase comprising the oil according to the invention.

3 designates an amphiphilic lipid.

4 designates a pegylated lipid.

5 designates an amphiphilic targeting ligand (or an amphiphilic [symbol] biovector [symbol] ).

3, 4 and 5 designate the compounds used as a surfactant in the nanoemulsion.

FIG. 2: Value of the average R2* of the tumor extracted from the R2* map, one hour after injection.

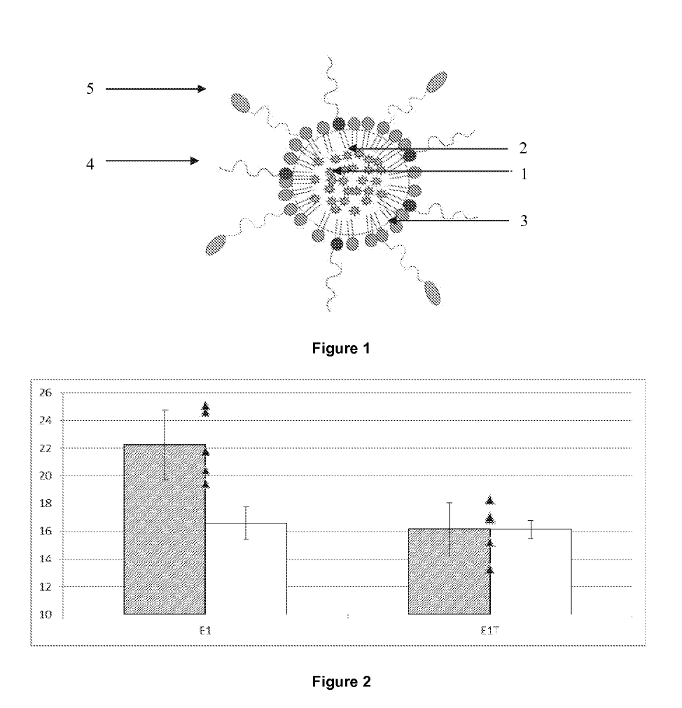

In hatched grey, appear the obtained average values of the R2* and their standard deviations, for tumors one hour after injection, either of the emulsion E1, or of its control E1T. In white, appear the obtained average values of the R2* and their standard deviations in the contralateral area to the tumor.

The black triangles correspond to the R2* points of the tumors.

### EXAMPLES

### General In the following, the abbreviations M, theoretical M, N and M/z, ES⁺, ES⁻, kD and TLC, Z ave,

XRD, Poly σ, RT, m_{obt} and Rdt have the following meanings:
M: molar concentration (mole/I).
Theoretical M: theoretical molecular mass.
N: normality.
M/z: mass over charge as determined by mass spectrometry.
ES⁺: positive mode electrospray.
ES″: negative mode electrospray.
kD: molecular mass unit (kilodalton)
TLC: thin layer chromatography
Z ave: hydrodynamic diameter measured by PCS in a unimodal mode
XRD=diameter of the particle (p) estimated by x-ray diffractometry
Poly σ: polydispersity measured by PCS.
RT: room temperature
m_{obt}: obtained product mass
Rdt: yield

### Dosage of Total Iron:

The iron concentration of the nanoemulsion composition is measured by inductively coupled plasma atomic emission spectroscopy (ICP-AES, Inductively Coupled Plasma Atomic Emission Spectroscopy), after mineralization in 65% HNO₃ (Optima™ 8300DV equipment from Perkin Elmer or equivalent).

### Size of the Particles/Droplets

### Hydrodynamic Diameter of the Nanoemulsion Droplets (Z ave)

Determined by PCS (Malvern Nano ZS apparatus, laser at 633 nm at 173°) on a sample diluted to ˜-1 millimolar with PPI water filtered on 0.22 μm.

PCS=Photon Correlation Spectroscopy: A technique by Dynamic Light Scattering—Reference: R. Pecora in J. of Nano. Res. (2000), 2, p 123-131.

### Diameter of the Magnetic Particle (p)

Determined by x-ray diffractometry (or XRD) by means of a curved INEL CPS-120 detector. The processing and measurement software package is Fityk®, the peaks are modeled according to [symbol] split Pearson VII [symbol] functions.

### Structural Analyses

For qualitative analysis, by mass spectrometry MALDI-TOF: a mass spectrometer coupling a laser ionization source assisted with a matrix (MALDI, matrix-assisted laser desorption/ionization) and a time-of-flight analyzer (TOF, time-of-flight mass spectrometry). The sample is thus diluted to 1/50 in tetrahydrofurane (THF), and then the mixture is diluted to 1/10 in the matrix (DHB=2,5-dihydroxybenzoic acid at 20 mg/ml in THF) before analysis with the Voyager DE STR from PerSeptive Biosystems or equivalent.

For quantitative analysis, by LC/UV/corona (liquid chromatography on an UltiMate3000 instrument (Dionex) coupled with the corona detector (Dionex)).

### Relaxivity Measurements

The relaxation times T1 and T2 were determined by standard procedures on a Minispec 120 apparatus (Bruker) at 20 MHz (0.47 T) and 37° C. The longitudinal relaxation time T1 was measured by using an inversion recovery sequence and the transverse relaxation time T2 was measured by a CPMG technique.

The relaxation rates R1 (=1/T1) and R2 (=1/T2) were calculated for different total metal concentrations (varying from 0.1.10⁻³ to 1.10⁻³ mole/l) in an aqueous solution at 37° C. The correlation between R1 or R2 depending on the concentration is linear, and the slope represents the relaxivity r1 (R1/C) or r2 (R2/C) expressed in (1/second)×(1/mmol/l) i.e. (mM⁻¹·s⁻¹.

### Dosage of the Amphiphilic Targeting Ligand RGD

The dosage of the amphiphilic targeting ligand was carried out by MALDI-TOF mass spectrometry: a mass spectrometer coupling a laser ionization source assisted with a matrix (MALDI, matrix-assisted laser desorption/ionization) and a time-of-flight analyzer (TOF, time-of-flight mass spectrometry). The sample was first diluted to 1/50 in tetrahydrofurane (THF), and the mixture is then diluted to 1/10 in the matrix (DHB=2,5-dihydroxybenzoic acid at 20 mg/ml in THF) before analysis with the Voyager DE STR from PerSeptive Biosystems or equivalent.

### Example 1

### Preparation of Magnetic Nanoparticles (p) Covered with Oleic Acid

### 1. Synthesis of Magnetic Nanoparticles Based on Iron

A solution of 36 g (0.181 mol) of FeCl₂.4H₂O, 20 ml of 37% HCl in 150 ml of H₂O was introduced into a mixture consisting of 3 liters of water and 143 ml (0.302 mol) of 27% FeCl_{3.} 250 ml of 25% NH₄OH were rapidly introduced with strong stirring. The whole was stirred for 30 mins. The juices are removed by magnetic decantation. The ferrofluid was successively washed three times with 2 liters of water.

The ferrofluid was set under stirring for 15 mins with 200 ml of HNO₃ [2M], the supernatant was removed by magnetic decantation. The nitric ferrofluid was refluxed with 600 ml of water and 200 ml of Fe(NO₃)₃ [1 M] for 30 mins. The supernatant was removed by magnetic decantation.

The nitric ferrofluid was washed three times with 3 liters of acetone, and then was taken up with 400 ml of water. The solution was evaporated in vacuo down to a final volume of 250 ml.

| Concentration M/L | Z ave nm | Poly σ | XRD diameter |
|---|---|---|---|
| 4.85 | 40 nm | 0.22 | 8.0 nm |

### 2. Coating of the Magnetic Nanoparticles Based on Iron

46 ml of the solution of magnetic nanoparticles ([Fe]=1.14 Mu i.e. 7.24.10⁻⁴ M) prepared as indicated above, were diluted in 500 ml of NaOH (5.10⁻³ M). To this solution, 60 g of oleic acid, i.e. 212.10⁻³ M (292 molar equivalents) were added. The whole was rapidly stirred for 1 hr at room temperature and then decanted on magnetized plates. The flocculate was washed three times with 500 ml of acetone, if the latter was not subsequently solubilized in oil, and stored as a suspension in acetone.

### Example 2

### Synthesis of an Amphiphilic Targeting Ligand, for Which the Targeting Ligand is a RGD Peptidomimetic; Exemplary Naphthyridine Compound

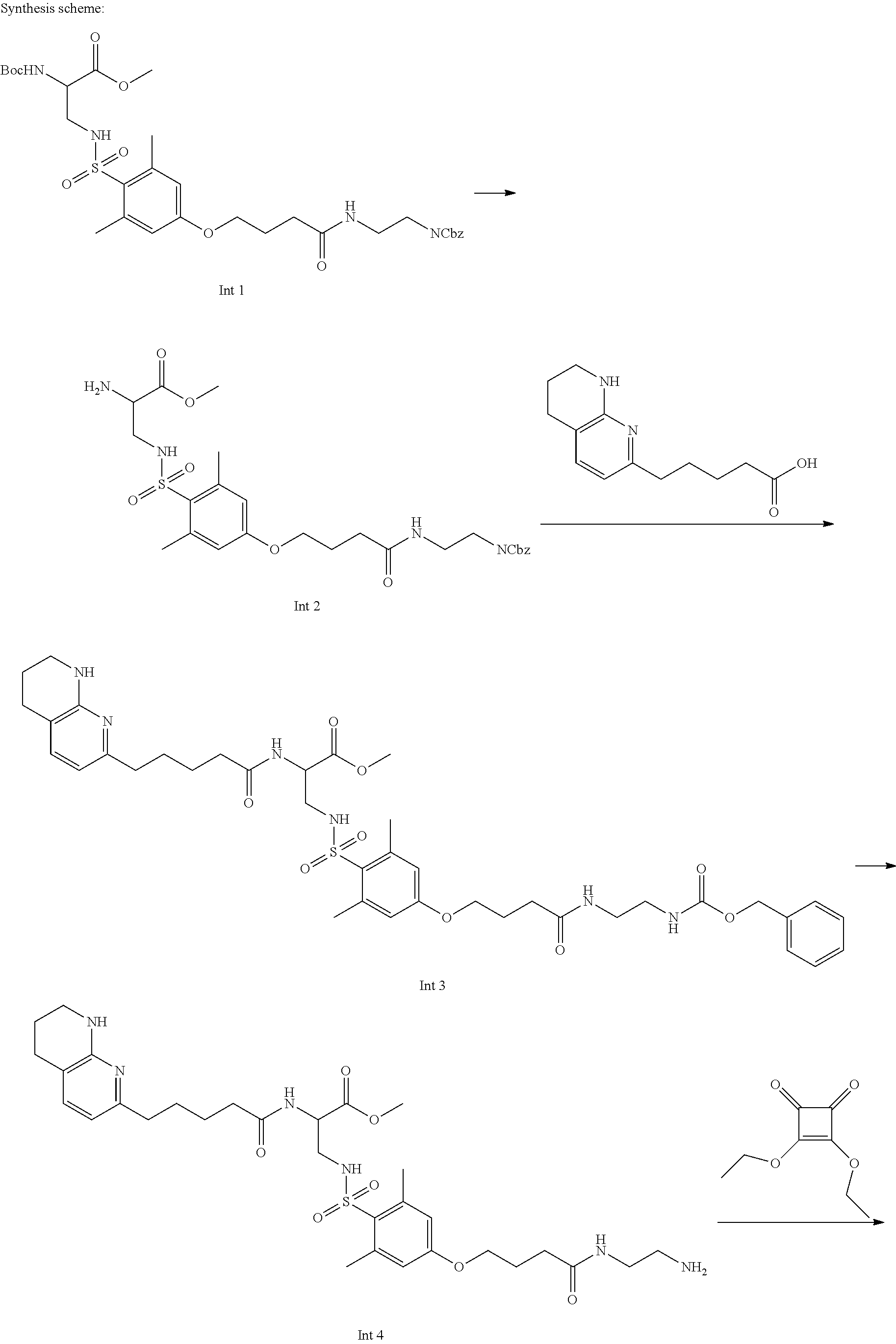

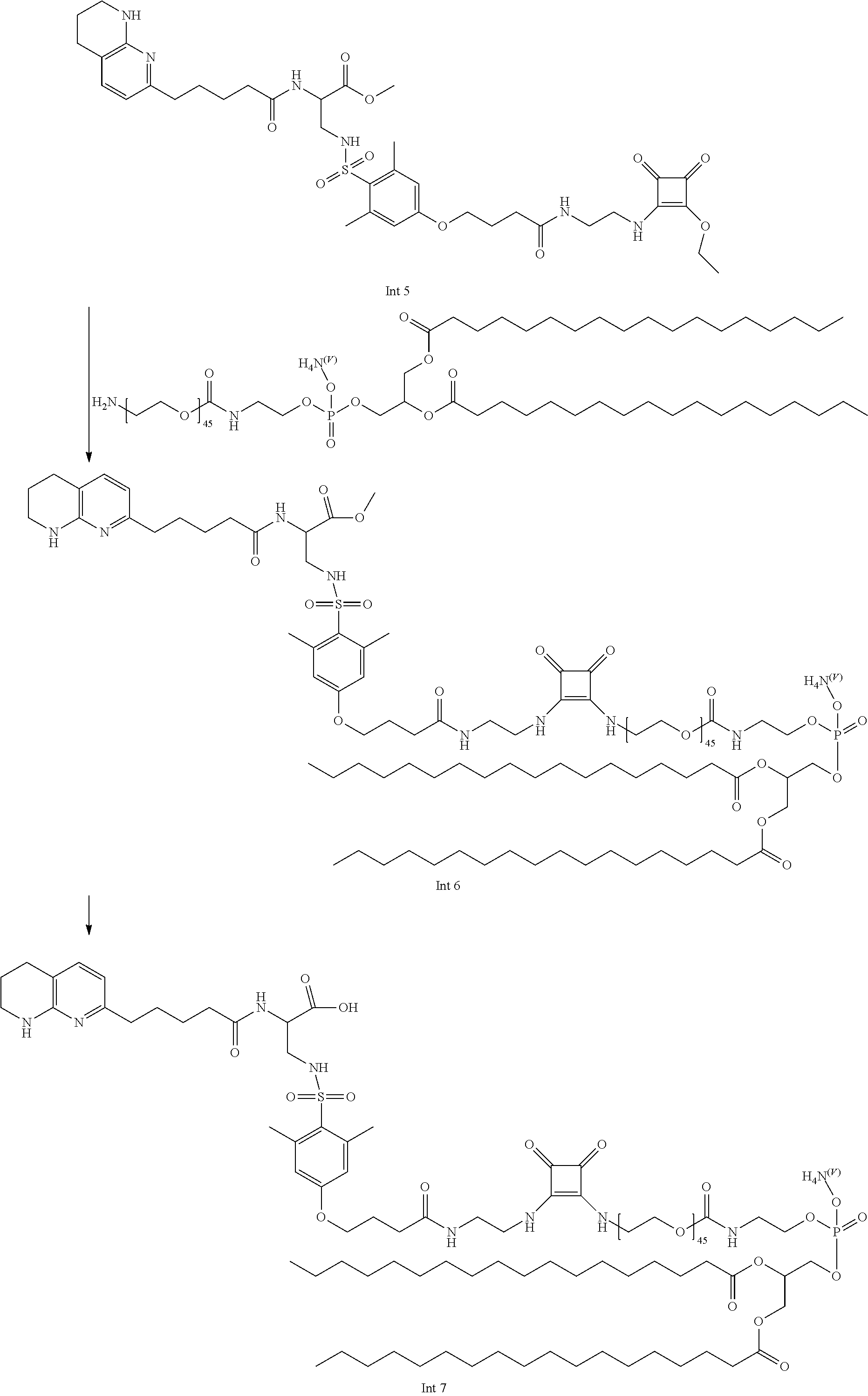

### Step 1

One gram of int 1 was dissolved in 5 ml of CH₂Cl₂. 5 ml of TFA were added to the medium. They were left for 3 hrs at RT and then dry evaporated. They were taken up in 2×40 ml of iso ether and then an oil was recovered which was dried by evaporation.

m_{obt}=0.8 g; Rdt=90%; C₂₆H₃₆N_{4O8}S; MALDI-TOF: positive mode m/z=564

### Step 2

| Reagents | Amounts | Solvents |
|---|---|---|
| Int 2 | M = 0.564 g (0.001 ml) | DMF V = 10 ml |
| Tetrahydronaphthyridine | M = 0.235 g (0.00023 ml) | |
| 2-pentanoic acid | | |
| HOBT | M = 0.131 g | |
| DIPEA | M = 0.286 g | |
| EDCI | V = 0.2 ml | |

Int. 3

The acid was dissolved in DMF and then HOBT and EDCI were introduced and left for 1 hr under argon.

Int 2 and DIPEA were added; left for 18 hrs at RT under argon. After evaporation, the oil was taken up in CH₂Cl₂ and washed with a diluted solution of Na₂CO₃; after evaporation, an oil is obtained.

m_{obt}=0.600 g; Rdt=77%; C₃₉H₅₂N₆O₉S; M/Z=780

### Step 3

| Reagents | Amounts | Solvents |
|---|---|---|
| Int 3 | M = 0.6 g (0.0077 ml) | MeOH V = 30 ml |
| Pd/C 10% | 1 spatula touch | |

Int. 4

Int 3 was dissolved in methanol and the solution was introduced into the 125 ml autoclave; the catalyst was added and left for 3 hrs under hydrogen pressure (P=5 bars) at 30° C. After filtration of the catalyst and evaporation, an oil was obtained which was washed with 50 ml of iso ether.

m_{obt}=0.300 g ; Rdt=60%; C₃₁H₄₆N₆O₇S; HPLC=90%; M/Z=646

### Step 4

| Reagents | Amounts | Solvents |
|---|---|---|
| Int 4 | M = 0.300 g (0.000442 ml) | DMSO V = 10 ml |
| Diethyl squarate | M = 0.286 g | TEA V = 0.25 ml |

Int. 5

Int 4 was dissolved in DMSO and then diethyl squarate and a few drops of TEA were added; the product is left overnight at room temperature under argon and is then poured into ether: a white slurry is then obtained.

m_{obt}=0.330 g; Rdt=97%; C₃₇H₅₀O₁₀S; M/Z=770

### Step 5

| Reagents | Amounts | Solvents |
|---|---|---|
| Int. 5 | M = 0.330 g (0.00043 ml) | DMSO V = 10 ml |
| DSPE-PEG₂₀₀₀-NH₂ | M = 1.07 g (0.000385 ml) | |
| Saturated Na₂CO₃ | M = 0.131 g | |
| solution | | |

Int 6 and Int 7

Int 5 and DSPE-PEG2000-NH₂ were dissolved in DMSO, 3 drops of saturated Na₂CO₃ solution and 2 ml of H₂O were added. The reaction medium was stirred at room temperature for 48 h and was precipitated from ether. The obtained slurry was solubilized in methanol and was then purified on silica, with CH₂Cl₂ eluent. After having collected and evaporated the compliant fractions: crystals were obtained.

Note: The obtained product is in the acid form by cleavage of the methyl ester by the presence of Na₂CO₃. Int 7 is therefore obtained directly.

m_{obt}=0.170 g; Rdt=17%; C₁₆₆H₃₀₈N₉O₆₃PS; M/Z=3,500

### Example 3

### Synthesis of an Emulsion Containing Magnetic Nanoparticles (p) of Example 1 and the Amphiphilic Targeting Ligand of Example 2

The magnetic nanoparticles based on an iron compound (synthesized in Example 1) were totally solubilized in 60 g of Miglyol® 812 oil at 92° C. for 20 hrs. Total solubilization of said magnetic nanoparticles was visually appreciated by noting the absence of any aggregate visible to the naked eye. After returning to room temperature, the solution was stored or used for making emulsions.

The surfactants (278.4 mg of Egg PC (Lipoid GmbH), 55.8 mg of DSPE-PEG-2000 (Lipoid

GmbH) and 27.9 mg of the amphiphilic targeting ligand of Example 2) were dispersed by means of ultrasonic waves into 20 ml of water with 2.5% mole/mole of glycerol.

5 g of Miglyol® 812 oil comprising the nanoparticles were added and pre-emulsified with the “Ultra-Turrax®” at a rate of 25,000 rpm (T 25).

The volume of the pre-emulsion, before microfluidization, was of about 25 ml. The pre-emulsion was then finalized with the microfluidizer (Microfluidics M-110-S) by recycling for 3 to 4 minutes at a pressure of about 1,200 bars, which corresponds to about 25 passages in the emulsion cell.

The emulsion was filtered over 0.45 p. The recovered emulsion volume was of about 22 ml.

### Characteristics of the Obtained Nanoemulsion

| Measured parameters | Results |
|---|---|
| Concentration of | 88 nM |
| nanoemulsion droplets | |
| Number of magnetic | 150 i.e. about 10⁶ Fe (a maghemite |
| particles per oil droplet | magnetic particle with a diameter of |
| | 8 nm has about 10,000 Fe molecules) |
| Fe concentration (mM) | 143 |
| Number of amphiphilic targeting | 3,500 |
| ligands (peptidomimetic RGD, | |
| compound of Example 2) per | |
| nanoemulsion droplet | |
| Hydrodynamic diameter (Z ave) | 190 nm |

This emulsion is called E1 in the following Examples.

### Example 4

### Relaxivity Measurements

The relaxivity measurements were conducted on Minispec® Relaxometers (Bruker Optics, Germany) at 60 MHz.

The mother solution was diluted over five range points in milli Q water in order to be able to study the linearity of the relaxation rates versus concentration. The range of concentrations ranged from 0.12 to 1.15 mM of Fe.

The relaxivity measurement was carried out at 37° C. The dosage of Fe was achieved by atomic emission spectroscopy on all the points of the range.

The results were the following:

60 MHz

| Emulsion | r1(mM⁻¹s⁻¹) | r2(mM⁻¹s⁻¹) |
|---|---|---|
| E1 (according to the invention) | 1 | 210 |

### Example 5

### Determination of the Stability of the Emulsion

The emulsion E1 according to Example 3 was tested as regards stability. Size measurements with PCS were conducted at 3, 6 and 9 months. The results are given in the following table.

| T 0 | | t = 3 months | | t = 6 months | | t = 9 months | |
|---|---|---|---|---|---|---|---|
| Z ave | | Z ave | | Z ave | | Z ave | |
| (nm) | Poly σ | (nm) | Poly σ | (nm) | Poly σ | (nm) | Poly σ |
| 193 | 0.059 | 192 | 0.043 | 191 | 0.06 | 187 | 0.064 |

### Example 6

### Toxicity Test of the Emulsion E1 of Example 3

### Test in Vivo

The following test in vivo was conducted:

On a “Swiss” mouse of about 25 g: Manual caudal vigil injection IV at 2 ml/min in an isovolume (200 μl/animal i.e. 6-7 ml/kg) and tracked for 14 days: measurement of hepatic, renal, hematological parameters at D1, D2, D7 and D14 and of histological parameters of the organs at D14.

At 24 h: anaesthesia with isoflurane, sublingual sampling for hematology results on the MS4 automatic apparatus and then exsanguination with syringe+heparin-coated needles.

Symptomatology: No ascertained lethality or deleterious clinical signs at the tested dose and delay.

Hematology: Normal hematological profiles.

Development of the weight of the mice after iniection: No significant weight variation.

Conclusion: After analyzing the results obtained in hematology as well as the various clinical and post-mortem observations of the mice, the conclusion may be drawn that there is no toxic effect of the nanoemulsion according to the invention. The histological examination of the organs did not either show any tissue abnormality.

### Example 7

### Measurement of the Affinity (IC50) of nanoemulsions according to the invention and of other non-compliant emulsions (comparative emulsions).

IC50 measurement of the emulsions was conducted on HUVEC cells over-expressing αvβ3 by competition measurements with Echistatin¹²⁵I.

### Materials

The binding buffer had the following composition: 20 mM of Tris (pH 7.4), 150 mM of NaCl₂, 1 mM of MgCl₂, 1 mM of MnCl₂, 0.5% of BSA.

Echistatin (MW=5417.12 Da) was provided by Bachem in freeze-dried form (ref. H-9010-100 μg). It was taken up at 1 mg/ml in water/0.1% TFA.

The iodinated precursor, ¹²⁵I-SIB has an activity of 2 mCi/mol. The characteristics of the phosphate buffer and of the borate buffer were respectively the following: 10 mM pH 7.2 and 0.53M pH 8.5. Bovine albumin, acetonitrile and trichloroacetic acid (TCA) were provided by Sigma.

To the compound ¹²⁵I-SIB (1,400 μCi-551.8 MBq) coated in a glass tube were successively added 15 μg of echistatin (15 μl) and borate buffer (final 0.2 M). This reaction mixture was incubated for 30 min with stirring, at room temperature. The coupling yield was determined by TLC in 10% TCA. The marking solution was then purified by a filtration gel on a PD10-Sephadex G25 column saturated beforehand with 0.5% BSA/PBS.
The characteristics of the echistatin-¹²⁵ISIB solution were the following:

C=1.245 μM-6.751 μg/ml

AS=16.02 μCi/μg

The tested products are shown in the following table.

### Characteristics of the Tested Products

| | Nanoemulsion droplet | Iron |
|---|---|---|
| Product | concentration (nM) | concentration (mM) |
| E1T | 95 | 127 |
| E1 | 88 | 143 |
| E1′ | 83 | 140 |
| E1″ | 82 | 138.6 |
| E1′″ | 79 | 141.3 |
| E2 | 209 | 150 |
| E3 | 115 | 129 |
| E3T | 120 | 131 |

### Range of Tested Concentrations (in Moles of Nanoemulsion Droplets)

| | |
|---|---|
| Echistatin | 3 μM to 0.027 nM |
| E1T | 31.67 nM to 1 aM* |
| E1 | 88 nM to 3 aM |
| E1′ | 80 nM to 3 aM |
| E1″ | 82 nM to 3 aM |
| E1′″ | 82 nM to 3 aM |
| E2 | 69.67 nM to 1 fM |
| E3 | 115 nM to 3 aM |
| E3T | 120 nM to 3 aM |

*aM: means attomolar (10⁻¹⁸ M)
**fM: means femtomolar (10⁻¹⁵ M)

The emulsions E1T, E1′, E1″, E1′″, E2, E3 and E3T were prepared in the same way as the emulsion E1 according to the invention but with the following differences:
E1T and E3T are without any amphiphilic targeting ligand prepared according to the procedure of Example 2.
E1′, E1″ and E1′″ mainly differ from the emulsion E1, as regards their percentage of amphiphilic targeting ligand prepared according to the procedure of Example 2.
in E2, the Miglyol® oil was replaced with coconut oil.
in E3 and E3T, the Miglyol® oil was replaced with soybean oil.

The coconut oil enters the definition of the lipid oils according to the invention (percentage of C6-C18 saturated fatty acids was 14%). On the other hand, the soybean oil does not enter this definition (percentage of C6-C18 saturated fatty acids was 14%).

The soybean oil has the following composition

| Nature of the fatty acids of the | |
|---|---|
| fatty acid glycerides | Concentrations (in %) |
| Palmitic acid (C16:0) | 10% |
| Stearic acid (C18:0) | 4% |
| Oleic acid (C18:1 n-9) | 23% |
| Linoleic acid (C18:2 n-6) | 51% |
| Alpha-linolenic acid (C-18:3 n-3) | 7-10% |

The HUVEC cells were cultivated and amplified in an EBM-2 medium, enriched with EGM-2 additives (Lonza). Before conducting the affinity measurements, the HUVEC cells were treated with PMA in order to generate over-expression of the integrin αvβ3. At the end of this treatment, the cells were trypsinated and a cell suspension with 4.10⁶ cells/ml in a binding buffer was obtained.

### Methods

The suspension of the HUVECs was distributed into a 96-well plate with a conical bottom, in an amount of 2.10⁵ cells in 50 μl in a binding buffer. Fifty μl of the solutions with increasing echistatin concentration or products were added per well. The positive control was made by adding binding buffer without any competitor. The whole of the concentration points was produced in duplicate. The plate was incubated for 2 hrs at room temperature with stirring. Fifty μl of the echistatin-¹²⁵I-SIB solution at 3 nM were then distributed in each well and the plate was again incubated for 2 hrs at room temperature with stirring. The reaction mixtures were transferred in ampules containing 200 μl of a density cushion consisting of paraffin and of dibutyl phthalate (10/90). The microtubes were then centrifuged at 12,000 rpm for 3 mins. The tubes were finally frozen in liquid nitrogen, and then severed in order to count the cell sediment and the supernatant with the gamma counter. A competition curve was then plotted where the relative binding of echistatin¹²⁵I-SIB was determined by the following equation:

$$\frac{Set radioactivity in the presence of a competitor ( cpm )}{\begin{matrix}Relative binding of echistatin - {I}^{125} - \\ SIB = Radioactivity of the controlled sample ( cpm ) \times 100\end{matrix}}$$

The data were analyzed by means of the software package GraphPad Prism® 5.0 which determines the IC₅₀ values for each product from the competition curve.

### Results

| | % of amphiphilic targeting | |
|---|---|---|
| | ligand based on the total | IC50 (pM of |
| | amount of surfactant | nanoemulsion |
| Tested product | (mole/mole) | droplets) |
| Echistatin | — | 700 |
| E1T control | 0 | 861 000 |
| E1 (according to the | 2 | 2 |
| invention) | | |
| E1′ (according to the | 1 | 4 |
| invention) | | |
| E1″ (according to the | 0.2 | 12 |
| invention) | | |
| E1′″ (according to the | 0.05 | 91 |
| invention) | | |
| E2 (according to the | 1 | 33.18 |
| invention) | | |
| E3 | 1 | 2278 |
| Control E3T | 0 | 120 000 |

Very good affinity for the αvβ3 receptor of the USPIO emulsions according to the invention is observed. The affinity of the emulsion E1 comprising 2% (mole/mole based on the total amount of surfactant) of amphiphilic targeting ligand is clearly larger than that of the control (E1T), by about 4 to 5 log and has a dose/response relationship depending on the RGD amphiphilic targeting ligand content.

On the other hand, an emulsion for which the lipid phase comprises an oil not compliant with that of the present invention (in this case soyabean oil), does not give a satisfactory result in terms of affinity. It is three times less affine than echistatin.

### Example 8

### MRI Results

### A—Study of the Specificity of the Targeting of the Glioma

### 1. Material and Method

a) Tested Products:
Emulsion E1 ([Fe]=143 mM)
Emulsion E1T ([Fe]=127 mM)

b) Cell Culture

The cells U87-MG (cell line of a human glioblastoma available at ATCC) were cultivated at 37° C. under a 5% CO₂ and 95% air humid atmosphere in a low glucose DMEM culture medium supplemented with 10% of inactivated newborn calf serum and 1% of glutamine.

The cells for intracerebral injection were obtained by trypsination of confluent cells and resuspended in sterile PBS at a concentration of 1,108 cells per ml.

c) Tumoral Induction

The xenograft was carried out on six week old nmri/nude mice. The mice were anaesthetized by intraperitoneal injection of 10 ml/kg of a mixture of Imalgene 1,000, Rompun 2% and saline. After local anaesthesia with Xylovet and disinfection with Betadine, the cancer cells were injected into the caudate nucleus according to a technique known to one skilled in the art. The animal was then placed in an incubator until it awakes.

d) Infections The products to be tested were injected at the dose of 100 μmol/kg.

For each mouse, a high resolution 3D map was produced post injection of the product. This gave the possibility of obtaining a map in 46 to 69 minutes.

e) Imaging in Vivo

An MRI was produced 21 days after implantation of the cells in order to locate the presence of a possible tumor induced in the animals, measure their size and thus select the animals having a sufficiently large tumor (i.e. a greater diameter of 3 to 5 mm) in order to be subject to imaging with injection of one of the contrast agents assigned to the mice before induction.

A Bruker 2.35 Tesla imager was used. The mice were anaesthetized with Isoflurane and maintained at 37° C. and then placed in a quadrature MRI probe (Rapid Biomed).

f) Treatment of the Obtained Images and Quantitative Analysis

The post-injection images were analyzed by means of the image processing software package (“Post Processus Software”). The values R2* were compared with the healthy portion among the groups of animals.

### 2. Results

| | E1 | E1T |
|---|---|---|
| R2* average tumor | 22.3 | 16.2 |
| R2* average contra | 16.6 | 16.2 |
| R2* standard deviation tumor | 2.5 | 1.92 |
| R2* standard deviation contra | 1.2 | 0.62 |

The results, also shown in the form of a histogram in FIG. 2, shows an increase of R2* in the tumor relatively to the healthy portion during injection of the specific product.

According to the Shapiro-Wilk normality test, the data follow a normal law, which gives the possibility of using the test-t on the averages of the R2* in the tumor between the specific and non-specific product. The result is significant (p=0.001487).

The results obtained during this study go in the direction of a specificity for targeting integrin αvβ3 in vivo with the emulsion E1 at 2.35 Tesla and of possible differentiation one hour after injecting the product for a dose of 100 μmol/kg.

The emulsion E1 therefore actually gives the possibility of obtaining clinical data from which it is possible to draw the conclusion of the presence of a tumor in the body of a subject having received said product.

### B—Follow-Up of the Anti-Angiogenic Treatment with Avastin®

### 1. Material and Method

a) Product Used

The emulsion E1 was used (iron concentration=143 mM) at the dose of 200 μmol/kg i.e. 1.4 ml/kg.

b) Imaging in Vivo

The same imaging technique as for the study of specificity of the targeting of the glioma was used.

21 days post induction, the mice were selected on the size criterion of the tumor. Those for which the major axis was comprised between 3 and 5 mm were retained (this is time D0).

For 13 mice (7 treated with Avastin® and 6 with saline), the chronology was the following:

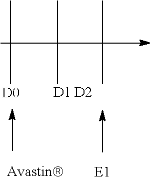

The imaging with the emulsion E1 was carried out 2 hours post injection.

c) Processing of the Images Obtained and Qualitative Analysis

The images two days after treatment and two days after injection were analyzed by means of an image processing software package (“Post Processus Software”). A qualitative analysis of the R2* maps on the whole of the tumoral volume was carried out. Four independent readers not aware of the administered treatment, classified the animals into two groups ( [symbol] treated with Avastin® [symbol] or [symbol] treated with saline [symbol] on the basis of R2* maps. From the four readers, the specificity and the sensitivity of the method for following the treatment were computed.

### 2. Results

| Reader | 1 | 2 | 3 | 4 | Average |
|---|---|---|---|---|---|
| Sensitivity (%) | 86 | 71 | 86 | 71 | 79 |
| Specificity (%) | 83 | 83 | 83 | 100 | 88 |

The experimental model was actually validated with a decrease of the expression of alpha V beta 3 in the animals treated with Avastin®.

The efficiency of the emulsion E1 in the follow-up of a treatment and notably of an anti-angiogenic treatment is thus demonstrated. This emulsion E1 allows a more reliable treatment follow-up than the other [symbol] biomarkers [symbol] which are the RECIST criterion, diffusion and Ktrans (existing comparison data but not shown).

1. An oil-in-water nanoemulsion composition, comprising:
50 to 90% by weight of aqueous phase;
9.5 to 49.5% by weight of lipid phase nanodroplets, wherein the lipid phase nanodroplets comprise an oil and magnetic particles, wherein the oil comprises at least 70% by weight of C6-C18 saturated fatty acid glycerides, and wherein the magnetic particles comprise an iron compound and are covered with one or more C8-C22 fatty acids; and
0.38 to 4.95% by weight of a mixture of surfactants at the interface between the aqueous and lipid phases, wherein the mixture of surfactants comprises at least one amphiphilic lipid and at least one amphiphilic targeting ligand, and wherein the mixture of surfactants is 4 to 10% by weight of the oil;
wherein the composition comprises more than 100 mmol of iron per liter of composition.

2. The composition according to claim 1, wherein the amphiphilic targeting ligand is 0.01 to 10% mole/mole of the total amount of surfactants.

3. The composition according to claim 1, wherein the saturated fatty acid glycerides are saturated fatty acid triglycerides.

4. The composition according to claim 1, wherein the at least 70% by weight of C6-C18 saturated fatty acid glycerides is at least 70% by weight of C6-C14 fatty acid glycerides or at least 70% by weight of C8+C10 fatty acid glycerides.

5. The composition according to claim 1, characterized in that the oil comprises a mixture of diglycerides and/or triglycerides of one or several fatty acids selected from caprylic acid, capric acid, linoleic acid, succinic acid, and a methyl, hydroperoxyl, hydroxyl, oxoyl, epoxyl, methoxyl, halogenated, amine, cyanyl, nitrosyl or thiol derivative of the foregoing.

6. The composition according to claim 1, wherein the iron compound is maghemite, magnetite or a mixture thereof.

7. The composition according to claim 1, wherein the magnetic particles are superparamagnetic.

8. The composition according to claim 1, wherein the mixture of surfactants comprises 80 to 96.95% mole/mole of amphiphilic lipid, 3 to 15% mole/mole of pegylated lipid, and 0.05 to 5% mole/mole of amphiphilic targeting ligand.

9. The composition according to claim 1, wherein the amphiphilic lipid is a phospholipid.

10. The composition according to claim 1, wherein the amphiphilic targeting ligand has the formula Bio-L-Lipo, wherein Bio is the targeting ligand localized at the external surface of the nanodroplets, Lipo is a lipophilic group extending within the mixture of surfactants, and L is a binding group connecting Bio and Lipo wherein L is selected from the group consisting of:
a simple bond;
squarate;
C1-C6 alkylene;
polyethylene glycol; and
P1-I-P2, wherein P1 and P2 are independently selected from the group consisting of 0, S, NH, a simple bond, CO₂, NHCO, CONH, NHCONH, NHCSNH, SO₂NH—, NHSO₂—, and squarate; and wherein I is selected from the group consisting of: alkylene; alkoxyalkylene; polyalkoxyalkylene; alkylene interrupted with one or several squarates; alkylene interrupted with one or several aryls; alkylene interrupted with one or several groups selected from —NH—, —O—, —CO—, —NH(CO)—, —(CO)NH—, —O—(CO)—, and —(OC)O—; alkenylene; alkynylene; alkenylene interrupted with one or several groups selected from —NH—, —O—, —CO—, —NH(CO)—, —(CO)NH—, —O(CO)—, and —(OC)O—; and alkynylene interrupted with one or several groups selected from —NH—, —O—, —CO—, —NH(CO)—, —(CO)NH—, and —O(CO)—.

11. The composition according to claim 1, wherein the targeting ligand of the amphiphilic targeting ligand is selected from the group consisting of peptides, pseudopeptides, peptidomimetics, amino acids, agents for targeting integrins, glycoproteins, lectins, pteroic or aminopteroic derivatives, derivatives of folic and antifolic acid, antibodies or antibody fragments, steroids, oligonucleotides, sequences of ribonucleic acid, sequences of deoxyribonucleic acid, hormones, proteins, mono- or poly-saccharides, and compounds with a benzothiazole, benzofurane, styrylbenzoxazole/thiazole/imidazole/quinoline, or styrylpiridine backbone.

12. The composition according to claim 1, wherein the targeting ligand of the amphiphilic targeting ligand is a ligand of a target associated with angiogenesis.

13. The composition according to claim 1, wherein the amphiphilic targeting ligand has the formula: 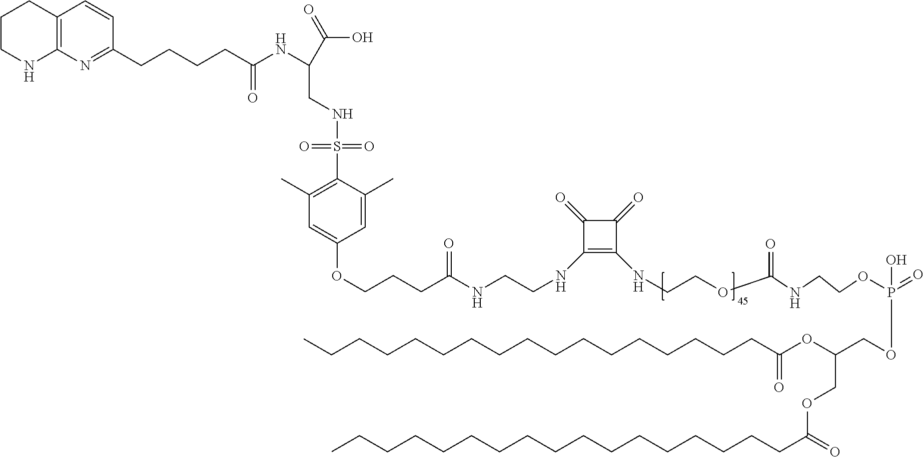
or one of its salts.

14. A method for preparing the oil-in-water nanoemulsion composition of claim 1, the method comprising the steps of:
a) solubilizing the magnetic particles in the oil to form the lipid phase; and
b) mixing the lipid phase and the aqueous phase, into which the mixture of surfactants is diffused, to form the lipid phase nanodroplets and thereby the oil-in-water nanoemulsion composition.

15. A contrast product comprising an oil-in-water nanoemulsion composition that comprises:
50 to 90% by weight of aqueous phase;
9.5 to 49.5% by weight of lipid phase nanodroplets, wherein the lipid phase nanodroplets comprise an oil and magnetic particles, wherein the oil comprises at least 70% by weight of C6-C18 saturated fatty acid glycerides, and wherein the magnetic particles comprise an iron compound and are covered with one or more C8-C22 fatty acids; and
0.38 to 4.95% by weight of a mixture of surfactants at the interface between the aqueous and lipid phases, wherein the mixture of surfactants comprises at least one amphiphilic lipid and at least one amphiphilic targeting ligand, and wherein the mixture of surfactants is 4 to 10% by weight of the oil;
wherein the composition comprises more than 100 mmol of iron per liter of composition.

16. The contrast product according to claim 15, wherein the targeting ligand of the amphiphilic targeting ligand is a ligand of a target associated with angiogenesis.

17. A method for obtaining one or several images of an entire body of an individual or a portion of the body of the individual, the method comprising performing a medical imaging technique on the entire body or the portion of the body of the individual, wherein said entire body or said portion of the body of the individual comprises the oil-in-water nanoemulsion composition according to claim 1 to obtain the one or several images thereof, wherein said image(s) is(are) associated with the magnetic particles of the oil-in-water nanoemulsion composition.

18. A method for evaluating angiogenesis in an individual comprising the steps of:
a) performing a medical imaging technique on the individual's entire body or of a portion of the individual's body, wherein said entire body or said portion of the body of the individual comprises the oil-in-water nanoemulsion composition according to claim 12, to obtain the one or several images thereof, wherein said image(s) is(are) associated with the magnetic particles of the oil-in-water nanoemulsion composition; and
b) viewing said images to evaluate angiogenesis in the entire body or the portion of the body of the individual.

19. A method for evaluating the efficiency of an anti-angiogenic treatment administered to an individual, the method comprising the steps of:
a) evaluating angiogenesis in the individual having received the anti-angiogenic treatment by conducting the method of claim 18; and
b) comparing the angiogenesis evaluated according to step a) with an angiogenesis reference from the individual from before the anti-angiogenic treatment was administered to the individual to evaluate the efficiency of the anti-angiogenic treatment.

20. (canceled)
